(19) United States
(12) Patent Application Publication
(10) Pub. No.: US 2023/0167403 A1
(43) Pub. Date: Jun. 1, 2023

(54) DROPLET ENCAPSULATION OF A CELL AND CONTROLLED RELEASE PARTICLE

(71) Applicants: THE REGENTS OF THE UNIVERSITY OF CALIFORNIA, Oakland, CA (US); PIONEER HI-BRED INTERNATIONAL, INC., Johnston, IA (US)

(72) Inventors: Abraham P. Lee, Irvine, CA (US); Gisela Lin, Irvine, CA (US); Yue Yun, Johnston, IA (US); Elizabeth Chatt, Johnston, IA (US); Marzieh Ataei, Irvine, CA (US); Jui-Yi Chen, Irvine, CA (US)

(21) Appl. No.: 17/922,908

(22) PCT Filed: May 4, 2021
(86) PCT No.: PCT/US2021/030614

§ 371 Date: Nov. 2, 2022

Related U.S. Application Data
(60) Provisional application No. 63/019,766, filed on May 4, 2020.

(51) Int. Cl. C12N 5/00; C12N 5/04

(52) U.S. Cl. CPC C12N 5/0012; C12N 5/04

(57) ABSTRACT

Microfluidic devices and methods for co-encapsulation of a cell and a controlled release particle in one droplet are escribed herein. The devices and methods utilize laminar flow, high shear liquid-liquid interfaces, hydrodynamic vortices, and/or acoustic focusing to increase co-encapsulation efficiency. The precise variation of the droplets microenvironment is enabled by the controlled release particle co-encapsulated with the single cell in each droplet. This capability, coupled with established detection methods, provides an important tool for precise, single cell analysis.

### CROSS-REFERENCES TO RELATED APPLICATIONS

This application claims benefit of U.S. Provisional Application No. 63/019,766 filed May 4, 2020, the specification(s) of which is/are incorporated herein in their entirety by reference.

### STATEMENT REGARDING FEDERALLY SPONSORED RESEARCH OR DEVELOPMENT

This invention was made with government support under Grant No. IIP-1362165 awarded by the National Science Foundation. The government has certain rights in the invention.

### BACKGROUND OF THE INVENTION

The present invention relates to microfluidic encapsulation, namely, controlled release in a microencapsulated droplet containing a cell. This controlled release in a microencapsulated environment has applications in diverse fields of research and analysis, which include, but are not limited to, high-throughput screening of compounds within a microencapsulated droplet, conducting a consecutive series of chemical reactions within a microencapsulated droplet, creating a cascade of multi-omics detection within a microencapsulated droplet, and the analysis of controlled release factors related to cell embryogenesis or differentiation within a living cell within the microencapsulated droplet.

Precision cell and molecular platforms can be used in high-throughput screening, conducting chemical reactions, creating a cascade of multi-omics detection, and analyzing controlled release factors related to cell embryogenesis or differentiation within a living cell. However, these platforms have technological and economic challenges in key industries such as pharmaceuticals and agricultural biotechnology. Hence, there is a need for a novel platform that can be used in said applications.

Droplet-based microfluidic systems are compatible with many chemical and biological reagents, and capable of performing a variety of “digital fluidic” operations that can be rendered programmable and reconfigurable. However, current methods for single cell encapsulation in droplets are inefficient (˜1%) and waste precious, costly materials.

### BRIEF SUMMARY OF THE INVENTION

It is an objective of the present invention to provide a temporal controlled-release droplet platform that allows for active, controlled alteration of a droplet's microenvironment post-encapsulation, as specified in the independent claims. This capability, coupled with established detection methods, provides an important tool for precise, single cell analysis that can reveal new biological mechanisms and responses at unprecedented sensitivities and fidelities that can be studied, tuned, and utilized. Embodiments of the invention are given in the dependent claims. Embodiments of the present invention can be freely combined with each other if they are not mutually exclusive.

In some aspects, the present invention features controlled release within a microencapsulated droplet. This invention can be used in diverse applications that include, but are not limited to, high-throughput screening of compounds within a microencapsulated droplet, conducting a consecutive series of chemical reactions within a microencapsulated droplet, creating a cascade of multi-omic detection within a microencapsulated droplet, and analyzing cell-culture conditions. For example, the present invention may be used to study and optimize reaction efficiency of enzymatic or biochemical reactions in different reaction conditions, such as single cell RNA sequencing based on multiple-step protocols rather than one step. The present invention may also be used to analyze controlled release factors related to cell embryogenesis or differentiation within a living cell within the microencapsulated droplet, for instance, a transient induced by different transcriptional regulators responding to cell culture. Such controlled release factors may include controlled temporal release of a compound, different concentrations of a compound, different reagents, and various gene regulation factors, all of which may be evaluated for their effects on a living cell. Without wishing to be bound to a particular theory, the present invention can enable higher throughput studies of cell-level responses of more cell types, including those from precious samples, to a wider variety of compounds and/or other cells, while reducing waste and overall cost, leading to new medicines and improved crop yields.

According to some embodiments, the present invention features a method of modifying an environment of a cell in a droplet. The method may comprise encapsulating the cell and a controlled release particle containing a cargo in the droplet, and activating release of the cargo from the controlled release particle which changes the environment in the droplet. Preferably, the droplet remains intact during activation.

In other embodiments, the present invention features a method of analyzing an effect of a chemical on a cell. The method may comprise encapsulating the cell and a controlled release particle containing the chemical in a droplet, activating release of the chemical from the controlled release particle, and examining the effect of the chemical on the cell. Preferably, the droplet remains intact during activation.

According to other embodiments, the present invention features a high-throughput screening method comprising preparing a droplet library by encapsulating in one droplet a cell and at least one controlled release particle containing a chemical, activating release of the chemical from the controlled release particle in the droplets, and examining the effect of the chemical on the cell. Preferably, the droplets remain intact during activation. The method may be effective for screening a library of compounds.

In further embodiments, the present invention features a method of analyzing nucleotides in a microencapsulated droplet. The method may comprise encapsulating at least one cell and at least one controlled release particle containing one or more reagents for sequencing in the microencapsulated droplet, activating release of the one or more reagents from the at least one controlled release particle, extracting nucleotides from the at least one cell, and sequencing the nucleotides of the at least one cell. In a preferred embodiment, the droplet remains intact during activation.

High efficiency encapsulation can be achieved by strategically selecting the input particles (i.e. cells), particle concentration, droplet size (i.e. droplet diameter), and flow rates (which affect droplet generation frequency). Efficiency also varies with the flow rates of the dispersed and continuous phases, which are related to the in-channel fluidic pressure exerted by these phases.

In other embodiments, the present invention features a method to encapsulate single plant cells inside a hydrogel droplet. The hydrogel matrix may be natural or synthetic based. The vast versatility of hydrogels makes them the biomimetic material of choice for 3D cell culture applications. Moreover, hydrogels can be engineered to have desired mechanical properties, bioactive features, and adequate mass transport to achieve optimal tissue culture.

In other embodiments, the present invention features a method to trigger the release of reagents from droplets by the use of ultrasonic, photonic, or magnetic energy. The drug/reagent-containing droplets are co-encapsulated with plant cells in a larger droplet. Upon triggering, the reagent in the inner droplet can be released and react with the cell. The controlled release of inner droplet makes it possible to carry out multiple biochemical reactions at defined timepoints at single-cell level. While the ultimate goal is to break down the inner droplet, the goal in the first stage is to break the droplets in a bulk solution.

One of the unique and inventive technical features of the present invention is the controlled release particle co-encapsulated with a single cell in each droplet. Without wishing to limit the invention to any theory or mechanism, it is believed that the technical feature of the present invention advantageously allows for precise variation of the droplet's microenvironment by the controlled release particle. To the inventors' knowledge, the present invention makes it possible to perform a controlled release reaction in a single droplet, which is useful in high-throughput screening of compounds and assaying a cell's microenvironment, for example, analyzing RNA transcripts in a cell.

None of the presently known prior references or work has the unique inventive technical feature of the present invention. In 1-1-1 encapsulation, the prior arts aim to encapsulate a cell and a passive bead, such as a bar-coded bead, for indexing of cells to keep track of them as they are exposed to different chemicals via droplet fusion or through chemical reactions using compounds already in the droplet. The prior arts are also aiming for encapsulation of multiple cells to study cell-cell signaling and interaction. Controlled release particles are being designed for drug delivery, enhanced imaging, and other purposes where these particles are not encapsulated. These particles may be injected into patients at the site of need for systemic delivery or to eliminate overdosing. Therefore, the prior arts teach away the encapsulation of a controlled release particle with a cell.

Furthermore, one of ordinary skill in the art would not be motivated to try encapsulating the cells with the controlled molecular release particles because in the prior arts, activation of the controlled molecular release particles would cause the droplet to burst, therefore one of ordinary skill would not have an expectation of success. In contrast, the present invention is able to activate the release of the controlled release particles while maintaining the integrity of the droplet. The compounds in the particles are released over time according to a calibration curve, or released all at once due to triggered particle bursting/disintegration. Thus, the present invention allows for the microenvironment inside the droplet to be changed by the controlled release particle in a controlled manner while the droplet remains intact.

Generally, in vivo (mainly animal) studies are easier. The ultrasound machines have broader bandwidth and also larger areas of application. Biological cells are much more different from bubbles than droplets are. To get sufficient energy into the droplets on the chips to break the bubbles without disrupting the droplet interfaces is not trivial. It has both technical capabilities and fundamental limitations. Another limitation is the selectivity. For in vivo, one wants the drug to leak out and affect the surrounding (i.e. go into the cells or the tissue or even bind). In the droplets, one wants the drug to come out of the bubbles and only affect the cell next to it but not the droplet membrane surrounding it.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a top view schematic of high efficiency cell encapsulation microfluidic platform which can encapsulate up to two distinct samples in a single droplet for downstream analysis.

FIG. 1B shows an embodiment of droplet encapsulation of a single cell and a particle capable of controlled release. Cells of different types can be encapsulated in the droplets, exposed to particles with different release profiles that change the microenvironment inside the droplet accordingly.

FIG. 1C shows non-limiting examples of release profiles that can be achieved by particles in droplets, such as, for example, step function, linear, exponential, etc. Particles can be engineered to provide the desired profile.

FIG. 2A is a fluorescent image of Dil stained liquid droplets with lipid outer shell (i.e. “lipid vesicles”), an example of controlled release particles. Dil fluoresces brightly when inserted into a lipid membrane.

FIG. 2B shows 500 nm green fluorescence protein-tagged beads encapsulated in Dil-tagged lipid vesicles. Vesicle cargo could also be compounds to be released.

FIG. 2C is a diagram of a microfluidic device used to make lipid vesicles with molecular cargo. In this example, the cargo is DNA, but other molecules could be used. Lipid and DNA are simultaneously injected. Once inside the droplet, the cationic lipids and DNA vector start to mix instantly due to the chaotic advection resulting from the droplet moving in the rendering channel.

FIG. 2D is an illustration of a drug delivery microbubble where compounds are dissolved in the oil layer. Targeting ligands can be added to facilitate binding.

FIG. 2E shows a Brightfield image of drug delivery microbubbles. The gas core comprises nitrogen/perfluorocarbon (PFC) (N2/PFC). The outer shell comprises 1,2-distearoyl-sn-glycero Phosphocholine (DSPC) lipid monolayer and polyoxyethylene 40 stearate (Myrj 52)=PEG-40 stearate. Doxorubicin, a naturally fluorescent anthracycline antibiotic and anticancer drug, is dissolved in triacetin oil. The N2/PFC gas mixture helps to maintain bubble size by setting an osmotic equilibrium with water-soluble gases, enabling the PFC gas to resist the Laplace and outer fluid pressures. Lipid shell stabilizes the microbubble by maintaining the inner gas core, PEG-40 stearate emulsifier helps to prevent bubble coalescence.

FIG. 2F is a fluorescence image of microbubbles showing Doxorubicin fluorescence within the oil shell. Scale bar is 15 μm.

FIG. 3A shows an exploded view of one embodiment in the boxed section of FIG. 1A. Particles self-assemble along the channel wall while moving toward the high shear interface. At the droplet generation junction, both types of particles get pulled towards the high shear interface symmetrically from both channel boundaries, resulting in one particle of each type encapsulated in a droplet (dark grey=high shear region). This type of encapsulation is termed “1-1-1 encapsulation.”

FIG. 3B shows an exploded view of another embodiment in the boxed section of FIG. 1A. At the droplet generation junction, a relative shear imposed by a continuous phase on a dispersed phase results in three-dimensionally confined two independent vortices at the junction. Particles introduced from a first inlet are trapped in the first vortex while cells flowed in through the second inlet are trapped in the second vortex.

FIGS. 3C-3D show schematics of trapping mode and release mode, respectively, in the vortices. In FIG. 3C, the particles and the cells introduced from the inlets are initially trapped in the independent vortices. In FIG. 3D, the particles and the cells are later released by precisely controlling flow rate ratio/droplet size.

FIG. 3E is an alternative embodiment, a vibrating air cavity (e.g. trapped air bubble in liquid) which is positioned in the intersection region upstream from the orifice to disrupt particle-trapping vortices and facilitate cell/bead encapsulation.

FIG. 4 shows a schematic representation of a microfluidic device for agarose microgels generation. The right side of the schematic shows an expanded view of the box when the device is being operated.

FIG. 5A shows a snapshot of the microfluidic device during agarose microgels generation, corresponding to a flow rate of 2.5 μL/min and 4 μL/min, for the agarose solution and the oil phase, respectively. The orifice width is 200 μm for comparison.

FIG. 5B shows the collection of agarose microgels from the device to a vial containing the oil phase, via outlet tubing.

FIG. 6A shows the bright field image of the agarose hydrogels after resuspension in culture media.

FIG. 6B shows the fluorescent image of the agarose hydrogels after resuspension in culture media. The average diameter of the hydrogels is 326 μm.

FIG. 7A shows the gray scale image of the cell laden hydrogels after incubating with FDA. FIG. 7B shows the fluorescent image of the cell laden hydrogels after incubating with FDA. FIG. 7C shows the combined grayscale and fluorescent images which demonstrates successful FDA staining of the cells.

FIG. 8A shows an example of encapsulated maize microspores within embryogenesis promoting media. FIG. 8B shows an example of encapsulated canola microspores within embryogenesis promoting media. FIG. 8C shows an example of maize pollen within storage media exposed to liquid germination media where two of the three pollen grains have germinated.

FIGS. 9A-9C shows an encapsulated maize microspore after five days of culturing using bright field (FIG. 9A), Nile Red staining (FIG. 9B), and FDA staining (FIG. 9C) to illustrate the cell viability.

FIGS. 10A-10E show microfluidic droplet generation. FIG. 10A shows a microfluidic device for DED generation; FIG. 10B shows an image of droplet generation; FIG. 10C shows that DEDs are uniform in size (˜25 μm); FIG. 10D shows lipids (dissolved in the oil layer) are labeled with rhodamine; FIG. 10E shows DEDs encapsulating FITC in the inner phase.

FIG. 11A shows DEDs with 30 nm AuNPs in the inner core. Rhodamine-conjugated lipids (red) are used to visualize oil shell, and FITC (green) is encapsulated in the inner phase. DEDs before laser exposure show a bright oil shell (red) and an inner phase (green). FIG. 11B shows DEDs after laser exposure for 50 minutes, and that DEDs still remain intact. FIG. 11C shows DEDs with MNPs in the oil phase before exposure FIG. 11D shows MNPs-DEDs exposed to AMF at room temperature for 1 hour.

FIGS. 12A-12D show AuNPs-DEDs Before and after laser exposure.

FIG. 13 shows MNPs-DEDs Before and after AFM exposure.

FIG. 14 shows an illustration of DEDs functionalized with AuNPs using thiol-gold reaction

### DETAILED DESCRIPTION OF THE INVENTION

Following lists elements corresponding to a particular element referred to herein:
100 microfluidic device
102 cell
103 controlled release particle
104 droplet
106 dispersed phase fluid
107 flow stream
108 continuous phase fluid
109 high shear interface
110 combining channel
112 channel sidewall
114 first dispersed phase channel
116 second dispersed phase channel
117 aqueous phase channel
118 aqueous phase fluid
119 laminar interface stream
120 first continuous phase channel
130 second continuous phase channel
140 intersection region
145 droplet shearing junction
147 orifice
150 output channel
160 fluid flow controller
170 sorting module
180 vortex region
182 vortex

As used herein, the microfluidic devices employ fluid volumes on the scale of microliters (10⁻⁶) to femtoliters (10⁻¹⁵) that are contained within sub-millimeter scale channels. The structural or functional features may be dimensioned on the order of mm-scale or less, preferably in the micron scale or less. For example, a diameter or width of a channel or a dimension of an intersection or junction may range from <0.1 μm to greater than 1000 μm. In a non-limiting example, the microfluidic channel size may be at least 100 μm for plant cells. Alternatively, or in addition, a length of a channel may range from mm to greater than cm-scale. The microfluidic device may employ active or passive techniques for fluid transport and droplet production. Compared to the active approach, which fluid manipulation involves the use of micropumps and microvalves, the passive approach takes advantage of the characteristic flow field in microfluidics to control the interface and capillary instability, and consequently to produce droplets.

As used herein, the term “high shear interface” refers to a high velocity liquid-liquid interface formed between two immiscible liquids. Generally, the continuous phase flow rate is greater than the flow rate of the dispersed phase. For instance, the continuous phase flow rate may be about 2-5 times greater. At the aqueous-oil interface, the high continuous phase flow rate imparts the same velocity to the dispersed phase at the interface. Hence, the dispersed phase at the interface is at a higher velocity (shear) than the bulk.

As used herein, the term “laminar flow” refers to flow of a fluid, or fluids, in layers that do not mix. One of ordinary skill in that art would understand that at lower Reynold's numbers (<10), a laminar flow is established in the microfluidic channel. The fluid flows in parallel layers with no lateral mixing but with some minor diffusion.

As used herein, 1 cell and 1 particle can be encapsulated in 1 droplet with high efficiency (>30%). This type of encapsulation is termed “1-1-1 encapsulation.

As known to one of ordinary skill in the art, in a geometry-mediated regime, or squeezing regime, the droplet generation depends only on the size of the orifice and the flow rate ratio of the dispersed phase to the continuous phase flow rate, whereas interfacial tension and viscosity has no significant influence. The transition between the geometry-mediated regime to a dripping regime is dictated by the Capillary number (Ca),

$$C a = \frac{μ \cdot v}{σ} ,$$

where μ is the viscosity, ν is the velocity of the continuous phase, and σ is the interfacial tension between the two fluid phases. Generally, in the geometry-mediated regime, Ca is <10⁻¹. In the dripping regime, Ca may be ≥10⁻¹ and interfacial tension and viscosity can predict the formation of droplets.

Samples for Encapsulation

Particles

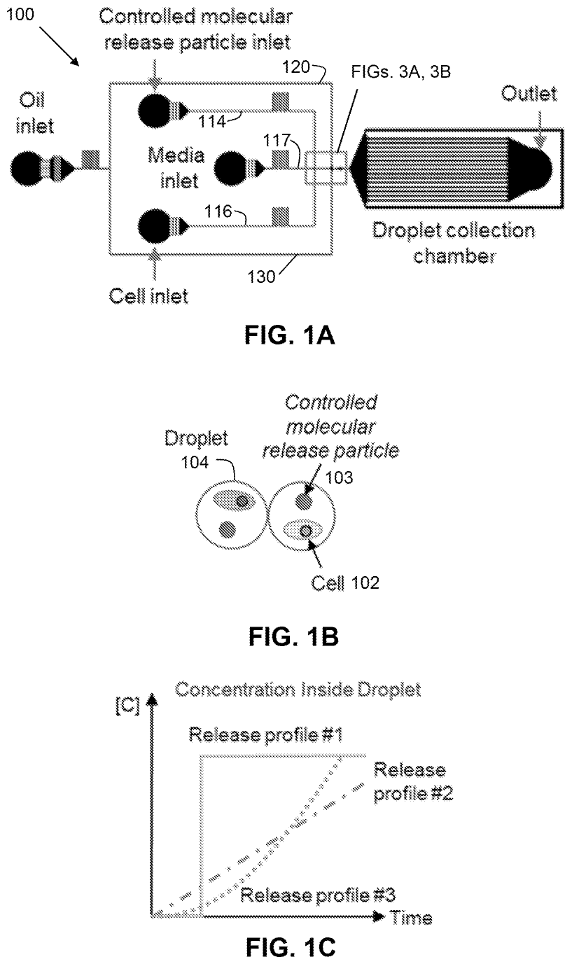

FIG. 1B shows samples encapsulated in a droplet. In some embodiments, the samples for droplet encapsulation may include microparticles (103), also referred to as particles. In some embodiments, the particles may have a size or dimension, such as a diameter or width, ranging from about 0.01 μm to about 20 μm. In other embodiments, the particle size or dimension is greater than 20 μm. In some embodiments, the microparticles may be solid, semi-solid, or core-shells. Examples of particles include, but are not limited to, disks, rods, and beads such as polymer beads, bar-coded beads, functionalized beads, and magnetic beads.

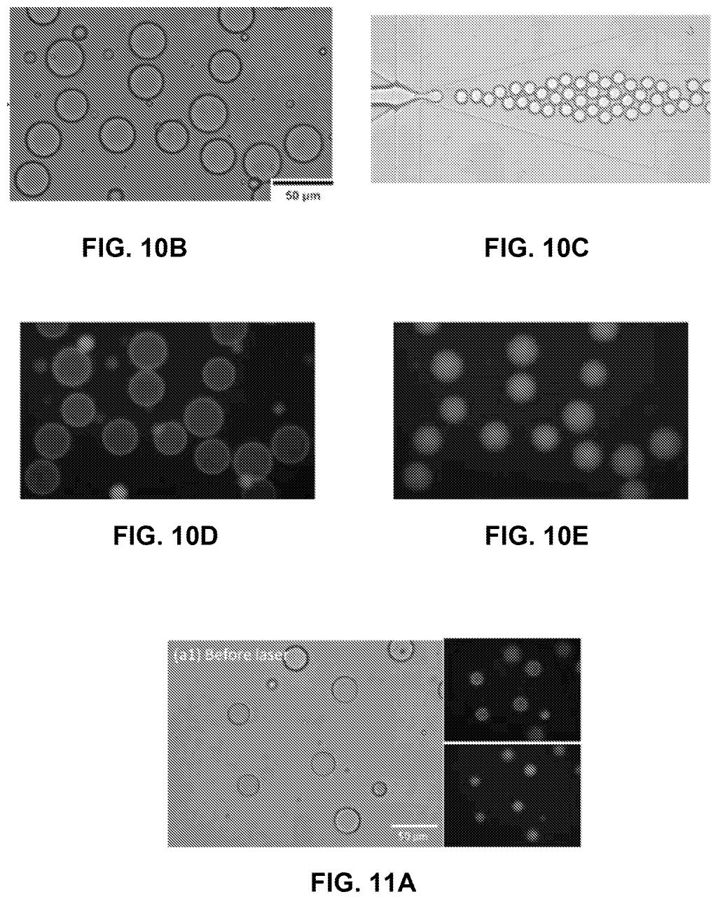

In preferred embodiments, the particles may be controlled release particles. Several forms of controlled release particles can be implemented with different release triggering mechanisms. Examples of such particles include, but are not limited to, solid core (bead), semi-solid core (polymer bead), liquid core (multi-layer droplet), and gas core (microbubble). Referring to FIG. 10, the controlled release particles may have varying release profiles. For example, the change in concentration of the released molecule may be linear, exponential, or step-wise.

As used herein, the term “cargo” refers to a chemical or item that is delivered to a cell or the environment of the cell or uptaken by the cell. The cargo can effect a change in the cell or the cell's environment or both. Non-limiting examples of cargo include, but are not limited to, drug compounds, reagents, cell media, nutrients, compounds for cell lysis, compounds that modulate cell growth, plasmids, nanoparticles, biomolecules, DNA, RNA, proteins, carbohydrates, or lipoplexes. The cargo may be a solid or a liquid. In some embodiments, the cargo may comprise one or more components. In some other embodiments, the cargo may be contained within a controlled-release particle, which is configured to release said cargo in some desired manner.

In some embodiments, the particles may be bar-coded or labeled. Bar-coding enables compound/cell indexing and identification of a release profile of a particle. For example, to keep track of which cells are exposed to which profiles, particles can be bar-coded beads or the droplets can be spatially indexed as they emerge from the droplet generation junction. In other embodiments, bar-coding can be used either independently or coupled with the particles. For instance, if the drug conditions or concentrations need to be tracked for drug screening, then the barcode and conditions may be coupled. In an alternative embodiment, for temporal controlled conditions for chemical reactions or time release, all the particles contain the same components therefore the barcode may not need to be coupled with the conditions.

In other embodiments, the particles may be made of natural or synthetic polymers. Example of said polymers include, but are not limited to, polyalkylenes (e.g. polyethylenes), polyethers (e.g., polyethylene glycol), polycarbonates, polyamides, polyamines, polyesters, polyvinyl alcohols, polyurethanes, and polyacrylates. In other embodiments, the polymer is a carbohydrate such as cellulose, dextran, glycogen, starch, carrageenan, amylose, chitin, chitosan, algin, agarose and xanthan. In yet other embodiments, the polymer may be a protein or peptide. In some embodiments, the polymers can be hydrophilic or hydrophobic. In other embodiments, the polymers can be cationic, anionic, or neutral. Any of the polymers may be modified with one or more functional groups. Examples of functional groups include, but are not limited to, amines, thiols, polyethylene glycol (PEG), lipids or fatty acids, biotin, neutravidin, or carbohydrates.

In a non-limiting embodiment, the solid microparticles may be constructed from commercial plastics, such as polystyrene, and purchased commercially. These particles can also be functionalized to introduce compounds into a droplet. In another embodiment, semi-solid core particles (polymer bead) include alginate and agarose hydrogel where compounds can be embedded and then released, passively, over time.

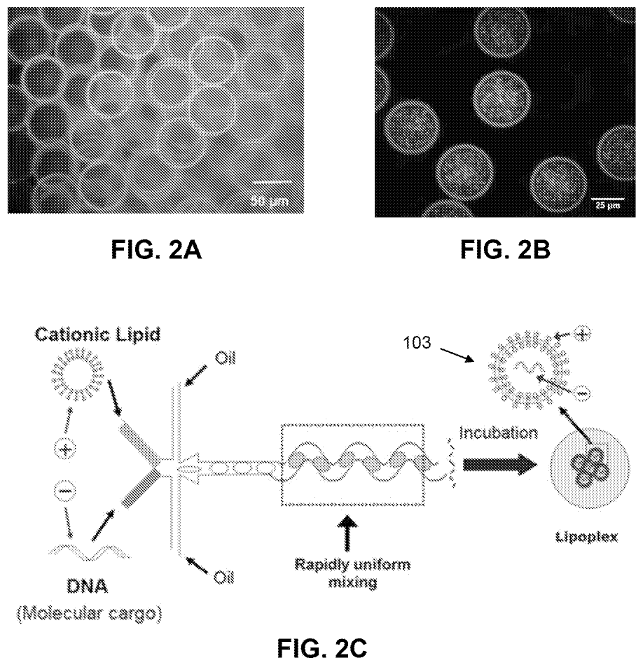

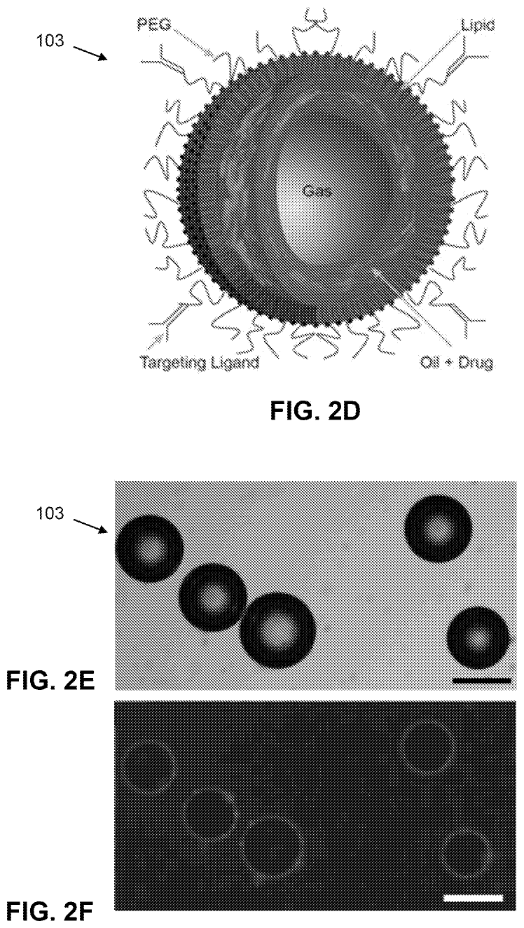

Referring to FIGS. 2A-2C, liquid core droplets may contain one or more outer shells. Compounds to be released can be contained in the one or more outer shells or contained in the internal core of the droplet. In FIGS. 2D-2F, multi-shell microbubbles comprise outer layers where at least one of the layers contains drug compounds. It is to be understood that while the particle may release drug compounds, the present invention is not limited to just drug compounds. In other embodiments, the molecules to be released from the particle may comprise reagents for nucleotide analysis, signaling compounds, cell media, nutrients, compounds for cell lysis, compounds that modulate cell growth, plasmids, nanoparticles, or lipoplexes and other chemical reagents or compounds.

In some embodiments, triggering mechanisms for controlled release can vary depending on the type of particle used. These mechanisms include, but are not limited to, physical properties such as light, pH, ultrasound, magnetism, temperature change, etc. For example, compounds in the outer layer of a microbubble may be released via external ultrasound. In other embodiments, a mechanism for controlled release may be physical contact. For example, the particle may release its contents when it contacts a cell. In some other embodiments, the controlled release particles may be modified using other molecules (linkers, etc.) that act as gateways to release. Further examples and details of controlled release particles can be found in US781160362, US20080166720A1, US20110045095A1, US20090098168A1, and US20110014297A1, the specifications of which are incorporated herein in their entirety by reference.

Cells

Referring back to FIG. 1B, in other embodiments, the samples for encapsulation may include cells (102). Any particular cell type from any organism may be used in the methods and systems of the present invention. The cells may have a size or dimension, such as a diameter or width, ranging from about 0.1 μm up to about 150 μm. In another embodiment, the size of the cell may be greater than 150 μm. In some embodiments, the cells may be wild type cells or genetically modified cells. In other embodiments, the cells may be cells harboring one or more mutations, healthy cells, stem cells, diseased or unhealthy cells, etc. For example, in some embodiments, the cells may be prokaryotic cells (e.g., bacteria, archaebacteria, etc.). In other embodiments, the cells may be eukaryotic cells such as single-celled eukaryotes, fungal cells (e.g. yeast, mold, etc.), animal cells, mammalian cells (e.g. cells from a human, non-human primate, rodent, rabbit, sheep, dog, cat, etc), and non-mammalian cells (e.g. cells from insects, reptiles, amphibians, birds, etc.).

In some embodiments, the cells used in the present invention may be other eukaryotic cells, such as plant cells or algal cells. Non-limiting and non-exhaustive examples of plant cells include cells from corn, soybean, wheat, cotton, grass, flowering plants, fruit-bearing plants, trees, tuberous plants, potatoes, root plants, carrots, peanut, nuts, beans, legumes, and squashes. It is to be understood that the term “plant cell” encompasses all types and stages of plant cells and is not limited to the aforementioned examples. Non-limiting and non-exhaustive examples of algal cells include cells from Chlorella sp., Nannochloropsis sp, and Botryococcus sp. It is to be understood that the term “algal cell” encompasses all types of algal cells and is not limited to the aforementioned examples. One of the distinguishing characteristics that plant and algal cells have over animal cells is a cell wall that surrounds a cell membrane to provide rigidity, strength, and structure to the cell. The cell wall may be comprised of polysaccharides including cellulose, hemicellulose, and pectin. Similar to plant and algal cells, the fungal cells also have a cell wall, which may be comprised of polysaccharides including glucans, mannans, and chitin. In some embodiments, the size of the plant cell may range from about 10 μm to about 100 μm. In other embodiments, the size of the plant cell may be greater than 100 μm.

In some embodiments, the cells used in the present invention may be walled-plant cells. In other embodiments, the cells may be protoplasts, which are intact plant, bacterial or fungal cells that had its cell wall completely or partially removed using either mechanical or enzymatic means. In some other embodiments, the cells used in the present invention may be plant egg cells.

In yet other embodiments, the cells used in the present invention may be a tetrad. The term “tetrad” is used herein to refer to a single structure comprising four individual physically attached components. A “microspore” is an individual haploid structure produced from diploid sporogenous cells (e.g., microsporoyte, pollen mother cell, or meiocyte) following meiosis. A microspore tetrad refers to four individual physically attached microspores. A “pollen grain” is a mature gametophyte containing vegetative (non-reproductive) cells and a generative (reproductive) cell. A pollen tetrad refers to four individual physically attached pollen grains.

As used herein, the term “sample” refers to cells, particles, or both. In some preferred embodiments, the samples may comprise a combination of cells (102) and microparticles (103). For example, the samples that are encapsulated in a droplet may comprise a cell and a particle. In a preferred embodiment, the particle is a controlled release particle.

Droplet Encapsulation

High efficiency encapsulation can be achieved by strategically selecting the input particles, particle concentration, droplet size (i.e. droplet diameter), and flow rates (which affect droplet generation frequency). Efficiency also varies with the flow rates of the dispersed and continuous phases, which are related to the in-channel fluidic pressure exerted by these phases.

In preferred embodiments, the present invention utilizes a microfluidic device for “1 cell and 1 particle in 1 droplet” (1-1-1) encapsulation with high efficiency (>30%). In some preferred embodiments, the particle encapsulated with the cell in the droplet is capable of controlled release such that the microenvironment inside the droplet changes over time in a predetermined manner. For example, the particle is capable of controlled release of a specific compound or group of compounds. The response of the cell can then be monitored simultaneously and the data correlated to the time release profile.

In some aspects, droplets are generated in microfluidic devices by flowing a first liquid (e.g., water) through a first channel and a second liquid (e.g., oil) that is immiscible with the first liquid through channels intersecting the first channel. The first liquid flowing through the first channel (e.g., water) is broken up to form discrete droplets as a result of shear forces from the second liquid. The size of the generated first liquid droplets generated can depend on a variety of factors including velocity of the second liquid. For example, as the velocity of the second liquid is increased, the size of the first liquid droplets is reduced.

Referring now to FIG. 1A, in some embodiments, the present invention provides a microfluidic device (100) for droplet encapsulating. The device may comprise a first microfluidic channel network (105) having a first fluid (106), comprising dispersed samples, flowing therein at a first flow rate (ν_{d}), a second microfluidic channel network (125) having a second fluid (108) flowing therein at a second flow rate (ν_{c}), an intersection region (140) formed by the second microfluidic channel network (125) intersecting the first microfluidic channel network (105), and an output channel (150). Preferably, a droplet shearing junction (145) is formed within the intersection region (140) as the second fluid stream (108) intersects the first fluid (106). The droplet shearing junction (145) may have an orifice (147) that fluidly couples the intersection region (140) to the output channel (150).

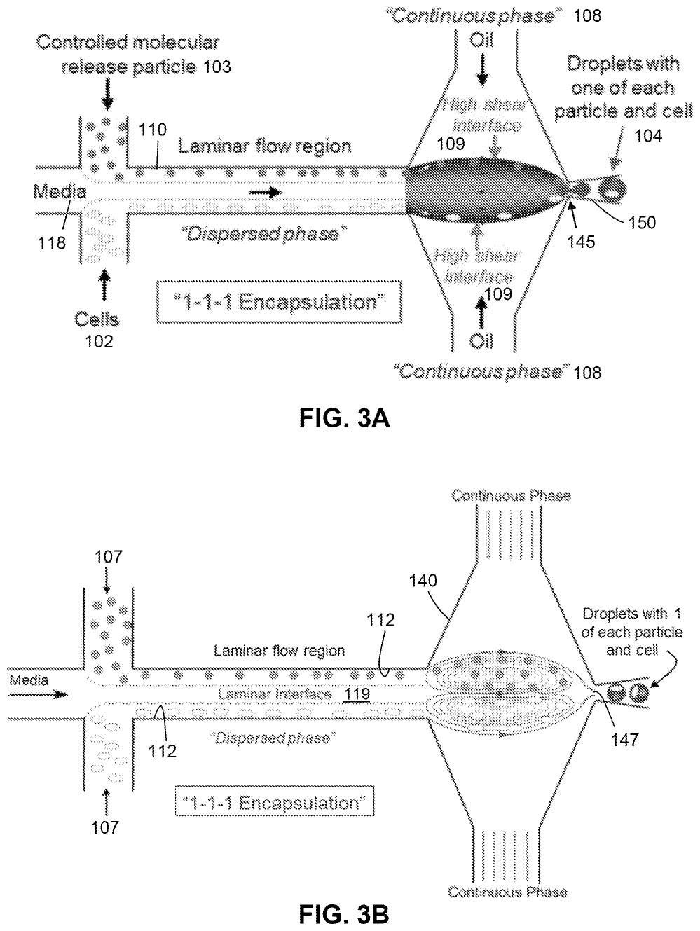

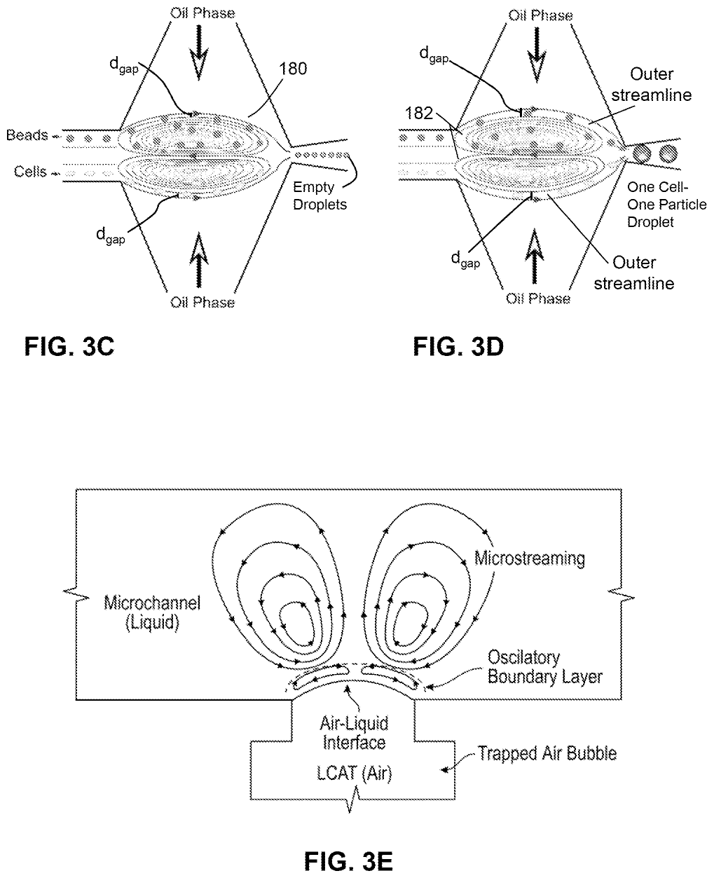

FIGS. 3A and 3B illustrate two different embodiments of the 1-1-1 encapsulation device that may be used in accordance with the present invention. Without wishing to limit the present invention, the encapsulation efficiency may depend on the cell concentration, droplet generation frequency, and droplet diameter. Efficiency also varies with the flow rates of the dispersed and continuous phases, which is directly related to the in-channel fluidic pressure exerted by these phases. Depending on what particle is used, parameters may need to be tuned to achieve optimal, repeatable 1-1-1 encapsulation with the cells. Cell and particle concentration, droplet generation frequency, droplet diameter, and flow rates of the dispersed and continuous phases are examples of said parameters that need to be optimized for the chosen controlled release particle.

In some embodiments, the device may further include a fluid flow controller configured to perform operations comprising adjusting ν_{d}, ν_{c}, or both to generate droplets (104) at the droplet shearing junction (145), which are outputted into the output channel (150). Preferably, said droplets (104) are substantially sized to co-encapsulate at least two different samples, for example, one cell (102) and one particle (103). In one embodiment, the flow in the microfluidic device (100) and adjustment of the flow rates may be pressure-driven. The microfluidic device (100) may utilize passive techniques to control fluid flow.

In one embodiment, the first microfluidic channel network (105) may comprise a plurality of inlet channels (115) merging into a combining channel (110). The first microfluidic channel network (105) may comprise 2-6 inlet channels (115). In some embodiments, each inlet channel (115) contains one flow stream (107) that flows into the combining channel (110). Preferably, the flow rate of the flow streams creates laminar flow in the combining channel (110).

In other embodiments, the second microfluidic channel network (125) may comprise a first continuous phase channel (120), and a second continuous phase channel (130). A portion of the first continuous phase channel (120) may be disposed on one side of the combining channel and a portion of the second continuous phase channel (130) may be disposed on an opposite side of the combining channel. The portions of the first and second continuous phase channels are configured to intersect the intersection region (140). For example, the portions of the first and second continuous phase channels can intersect at a terminal end of the combining channel to form the intersection region (140) to which the output channel (150) is fluidly coupled thereto. In one embodiment, the portions of the first and second continuous phase channels can intersect the combining channel (110) orthogonally such that the continuous phase channels and combining channel form a T-junction. Alternatively, the continuous phase channels can intersect the combining channel (110) at an acute angle such that the continuous phase channels and output channel form a Y-junction.

In some embodiments, the flow stream (107) through the inlet channels (115) may comprise dispersed samples or an aqueous phase fluid (118). For example, one or two inlet channels may have flow streams with dispersed samples and another inlet channel introduces aqueous phase fluid to the combining channel. In some preferred embodiments, the samples assemble near a sidewall (112) of the combining channel as they flow towards the intersection region (140). In one embodiment, the aqueous phase fluid (118) flows in the combining channel (110) such that the aqueous phase fluid (118) forms a laminar interface stream (119) between the dispersed sample flow streams. Non-limiting examples of the aqueous phase fluid (118) include water, lysis buffer, and/or other lysis reagents.

According to other embodiments, the microfluidic device may be used in a method for encapsulating a cell (102) and particle (103) in a droplet. The method may comprise flowing a first fluid (106), comprising dispersed cells (102) and particles (103), at a first flow rate (ν_{d}) through a first microfluidic channel network (105) and into an intersection region (140), and co-flowing a second fluid (108) through a second microfluidic channel network (125) at a second flow rate (ν_{c}). In one embodiment, the first fluid (106) may include at least two flow streams (107), one having cells (102) and the other having particles (103). Said cells and particles self-assemble near a sidewall (112) of the first microfluidic channel while flowing towards an intersection region (140). The first fluid (106) may also include a third flow stream for establishing laminar flow. In order to generate droplets, ν_{d}, ν_{c}, or both are further adjusted to generate droplets (104) at the droplet shearing junction (145), which are outputted into an output channel (150). Preferably, the droplets are substantially sized to co-encapsulate a cell and a particle.

Referring to FIG. 1B, in preferred embodiments, one cell (102) and one controlled release particle (103) may be encapsulated in one droplet (104). In alternative embodiments, multiple controlled release particles (103) may be encapsulated with the cell (102) in one droplet (104). The controlled release particle (103) may comprise a solid core, semi-solid core, liquid core, or gas core. In one embodiment, the particle (103) may be a bar-coded bead, shell, or bubble. In another embodiment, the particle may not be bar-coded. In some embodiments, release of the particle may be triggered by light, ultrasound, magnets, pH or temperature change. In other embodiments, release of the particle may be activated by physical contact with the cell. Non-limiting examples of chemicals or biomolecules molecules that may be released from the particle include RNA, DNA, reagents for nucleotide analysis, lysis reagents, drug compounds, signaling compounds, and other chemicals.

In some preferred embodiments, the sizes (e.g. width, length and height) of the various microfluidic channels, the intersection region, and orifice are sufficiently sized to accommodate plant cells.

In various embodiments, the width of the various microfluidic channels (e.g., the first and second dispersed phase and aqueous phase channels (114, 116, 117); the combining channel (110); and the continuous phase channels (120, 130)) can range from about 25 μm to about 500 μm. For example, the width of the various microfluidic channels can be about 30 μm to about 200 μm. As another example, the width of the various microfluidic channels may be about 100 μm to about 400 μm for plant cells.

In other embodiments, a width and/or length of the intersection region can be about 3-10 times the width of the various microfluidic channels (e.g., the combining channel, the first continuous phase channel, or the second continuous phase channel). For example, the width of the intersection region may be about 150 μm, which is about three times the width of a 50 μm incoming microfluidic channel. For plant cells, the width of the intersection region may be about 900 μm, which is about three times the width of a 300 μm incoming microfluidic channel. In another embodiment, the length of the intersection region may be about 1,200 μm, which is about four times the width of a 300 μm incoming microfluidic channel.

In some embodiments, the width of the orifice may be about 5-200 μm. For example, in one embodiment, the width of the orifice may be about 5-50 μm, about 50-100 μm, about 100-150 μm, or about 150-200 μm. In other embodiments, the width of the output channel may widen from the width of the orifice to a maximum width. The maximum width of the output channel can be about 2-10 times the width of the orifice. For example, for a 200 μm orifice, the output channel widens from a minimum width of 200 μm to a maximum width of about 500 μm. In further embodiments, the width of the output channel may be reduced after reaching its maximum. In some embodiments, the output channel splits into two or more channels. The width of said channels can range from about 25 μm to about 500 μm but are not necessarily the same widths.

In other embodiments, the height of the various microfluidic channels may be less than twice the diameter of the samples (e.g., cells, particles, etc.) that are in dispersed phase fluid. Restricting the height of the various microfluidic channels to be less than twice the diameter of the solid samples can advantageously reduce the chance that the solid samples roll over each other and/or stack over each other. For instance, the height of the microfluidic channels can range from about 25 μm to about 250 μm. For example, the height of the microfluidic channels may be about 50 μm to about 200 μm for plant cells.

It is to be understood that the present invention is not limited to any of the aforementioned dimensions. Furthermore, one of ordinary skill in the art can determine the appropriate dimensions of the microchannels and regions based on the type of cells and particles being encapsulated and/or the desired size of the droplets.

As previously described, it is an objective of the present invention to co-encapsulate a cell and at least one controlled release particle in one droplet. The following provides non-limiting examples of performing droplet encapsulation with high efficiency, e.g. >30%.

Interfacial Shearing

In FIG. 3A, the dispersed samples may comprise a plurality of cells (102) flowing in one of the flow streams (107) and a plurality of particles (103) flowing in another flow stream (107). When flowing through the combining channel (110), laminar flow causes the cells (102) to assemble near the sidewall (112a) and the particles (103) to assemble near an opposing sidewall (112b). The flow rates, ν_{d}, ν_{c}, or both, are adjusted such that the second fluid stream (108) forms a high shear interface (109) with the first fluid (106), and the solid samples are drawn to the high shear interface (109). At the intersection region (140), the cells are drawn to one high shear interface (109a) and the particles are drawn to an opposing high shear interface (109b), thus one cell and one particle can be co-encapsulated in one droplet (104) as said droplet (104) is formed at the droplet shearing junction (145). The droplet (104) co-encapsulating the one cell and one particle can then be released from the orifice (147) into the output channel (150).

Without wishing to limit the present invention, this method is a passive, hydrodynamic technique which can achieve a ‘one-one-one’ (one cell and one particle in one droplet) encapsulation efficiency of 30% or higher, which could significantly improve the biomolecular capture efficiency of various particle-based single cell applications.

The device can be configured to encapsulate one cell and one particle in a single droplet of a fluid (e.g., water) by the combined effect of laminar flow and the high shear liquid-liquid interfacial boundary. In the illustrated device of FIG. 3A, a first fluid stream comprising a first solid sample (e.g. cells or cellular material) dispersed in a first fluid (e.g., water) is introduced through a first incoming microfluidic channel and a second fluid stream comprising a second solid sample (e.g. particles) dispersed in the first fluid (e.g., water) is introduced through the second incoming microfluidic channel. A third fluid stream comprising the first fluid (e.g., water) is introduced through the third incoming microfluidic channel. The three flow streams, collectively referred to as a dispersed phase fluid stream, flow into the combining channel.

In some embodiments, the velocities of the first, second, and third flow streams can be adjusted such that laminar flow is established in the combining channel. For example, the flow rates of the first, second and third flow streams can be equal to each other such that laminar flow is established in the combining channel. By maintaining equal flow rates in the three incoming microfluidic channels, particle/cell migration across the streamlines due to Magnus force can be prevented. The first and the second fluid streams in the combining channel can be separated by a laminar interface as a result of the laminar flow. The constituents of the first solid sample (e.g., cells or cellular material) self-assemble on one side of the laminar interface and the constituents of the second solid sample (e.g., particles) self-assemble on another side of the laminar interface. For example, particles self-assemble in a single row along a channel wall of the combining channel adjacent to the incoming microfluidic channel of the particles, and cells self-assemble in a single row along the opposite channel wall of the combining channel adjacent to the incoming microfluidic channel of the cells. The laminar flow of the dispersed phase fluid stream enters the intersection region. In the intersection region, the flow rate of the continuous phase fluid streams can be adjusted to create a high shear interface between the laminar flow of the dispersed phase fluid stream. Cells in the first flow stream and the particles or particles in the second flow stream are pulled towards the high shear interface as shown in FIG. 3A.

In some embodiments, the flow rates of the dispersed phase fluid stream and the continuous phase fluid streams can be adjusted to generate droplets having a droplet size large enough to encapsulate a single cell from the first flow stream and a single bead/particle from the second fluid stream. The size of the droplet can depend on the capillary number, Ca=μV/σ, where μ is the viscosity of the continuous phase comprising the second fluid, V is the superficial velocity (flow rate) of the continuous phase comprising the second fluid, and σ is the equilibrium surface tension between the continuous phase and the dispersed phase fluid streams. To generate droplets having an appropriate size to encapsulate a single cell and single particle, the capillary number can be in the range of about 0.01 and about 1 (e.g., about 0.1). In various embodiments, the velocity of the continuous phase fluid streams can be about 2-10 times greater than the velocity of the dispersed phase fluid stream.

The droplet size can also be controlled by controlling the pressure ratio between the dispersed phase fluid stream and the continuous phase fluid stream. In various embodiments, a droplet encapsulating a single cell and a single particle can be achieved by controlling the pressure ratio (q) and/or the flow rate ratio between the dispersed phase and the continuous phase. In some embodiments, the pressure ratio and/or the flow rate ratio between the dispersed phase and the continuous phase may be about 0.1 to about 0.5 (e.g., about 0.3) in order to maximize encapsulation efficiency. Depending on the pressure ratio and/or the flow rate ratio, the generated droplets can be configured to have a diameter of about 20 μm to about 400 μm to match the size and/or concentration of the incoming cells and particles. For example, a plant cell and a controlled release particle may be encapsulated in a droplet having a diameter of about 300 μm to about 400 μm.

In various embodiments, the size of the generated droplets can be tuned by adjusting the droplet generation regimes. The encapsulation of a single particle and a single cell in a single droplet can be achieved in both geometry-mediated and dripping regimes. For example, when the droplets are generated in the geometry-mediated regime, the size of the droplet can be greater than or equal to the size of orifice diameter. In the dripping regime, where the droplet break-off occurs due to interfacial instability, the droplet size can be less than the size of the orifice. In both regimes, the particles and cells that assemble in a single row along the channel wall are pulled into the droplets by the symmetrical high shear zone resulting in encapsulation. The droplet size can be tailored to the size of the incoming cells and/or concentrations by controlling the pressure and/or flow rate ratio between the dispersed phase and the continuous phase and the capillary number. Without wishing to limit the present invention, the encapsulation efficiency achieved can be 30% or higher. Further details of interfacial shearing can be found in co-owned Application No. PCT/US2018/036952, the specifications of which are incorporated herein in their entirety by reference.

Hydrodynamic Vortices

Referring to FIG. 3B, in other embodiments, the continuous phase fluid (108) can intersects the dispersed phase fluid (106) to form a high shear interface (109) with the dispersed phase fluid. The intersection region may comprise a droplet shearing junction (145) formed as the continuous phase fluid (108) merges with the dispersed phase fluid (106), and a vortex region (180) comprising two vortices (182) each formed by one of the flow streams (107).

In one embodiment, the fluid flow controller (170) is configured to perform operations comprising adjusting ν_{d}, ν_{c}, or both to generate the vortex region (180), adjusting ν_{d}, ν_{c}, or both to trap and re-circulate the samples within the vortices (182), and adjusting ν_{d}, ν_{c}, or both to release the samples from the vortices (182) and generate droplets (104) encapsulating one cell (102) and one particle (103) at the droplet shearing junction (145). In one embodiment, the fluid flow controller (170) may comprise a feedback control system. The feedback control system may be configured to actuate a pressure controller to adjust the pressure to release the cells/particles if the intensity of the flow focusing junction exceeds a certain threshold. In some embodiments, intensity can be measured using an image processing module. In other embodiments, the pressure controller can be regulated using LabView program.

Referring to FIGS. 3C-3D, each vortex (182) can have an outer stream disposed between an outermost streamline of the vortex and the adjacent high shear interface. Each outer stream may be fluidly coupled to the orifice so as to be outputted into the output channel. The outer streams can have a maximum width, d_{gap}, between the outermost streamline of each vortex and the adjacent high shear interface. In one embodiment, d_{gap} can be reduced when adjusting the flow rates to trap and re-circulate the samples. In another embodiment, d_{gap} can be widened when adjusting the flow rates to release the samples from the vortices and into their respective outer stream for encapsulation at the droplet shearing junction. For example, when the cells and particles flow into the intersection region (140), the cells may be disposed in one vortex and the particles may be disposed in the other vortex. In a preferred embodiment, when adjusting the flow rate for release, one cell is released from its vortex into its outermost stream and one particle is released from its vortex into its outermost stream. The one cell and the one particle are then co-encapsulated in the one droplet (104) as said droplet is formed at the droplet shearing junction (145), and the droplet (104) co-encapsulating the one cell and one particle is released from the orifice into the output channel (150).

Without wishing to limit the present invention to a particular theory or mechanism, the occurrence of the microvortices in the aqueous/dispersed phase may depend on the combined effects of the aqueous-oil (dispersed phase-continuous phase) interfacial shearing rate (α) and the tip oscillation frequency (TOF), defined as the interfacial oscillation frequency of the droplet generation tip. As used herein, an approximate range of a is about 1-200 [1/s], with a increasing as the regime changes from squeezing to dripping to jetting. In some embodiments, the range of a in the squeezing regime may be about 1-50 s⁻¹, the range of a in the dripping regime may be about 50-100 s⁻¹, and the range of a in the jetting regime may be about 100-200 s⁻¹. The protrusion and retraction of the droplet generation tip occurs each time a droplet is generated; therefore, the tip oscillation frequency (TOF) is equal to the number of droplets generated per second (f).

Both the parameters α and TOF can vary with droplet generation regimes such that they are lowest in the squeezing regime and highest in the jetting regime, which is dictated by the capillary number

$$( C a = \frac{μ {V}_{c}}{σ} ) ,$$

where μ is the viscosity of the continuous phase, V_{c} is the velocity of the continuous phase and σ is the interfacial tension between the phases. Again, without wishing to limit the present invention, the microvortices may start to occur at higher values of a and TOF. In the squeezing regime, which occurs at lower Ca (e.g., about 10⁻³), both the interfacial shearing rate (α) and the TOF, which may be about 50 Hz, are not large enough to generate the microvortices. However, at a higher Ca (e.g., about 10⁻¹) where the regime switches from squeezing to dripping, the microvortices are likely to occur because of the increase in a and TOF, for instance, the TOF may be greater than >2000 Hz.

Without wishing to be bound to a particular theory or mechanism, ν_{d}, ν_{c}, or both can be adjusted such that a ratio of ν_{d} to ν_{c} reduces d_{gap} to be less than half a diameter of the samples, thereby trapping, re-circulating, and accumulating the samples within the vortices, and further reducing a width of the orifice to prevent encapsulation. In another embodiment, ν_{d}, ν_{c}, or both can be adjusted such that the ratio of ν_{d} to ν_{c} increases d_{gap} to be about ½-1.5 the diameter of the samples, thereby releasing the samples from the vortices into the outer streams, and further increasing the width of the orifice to allow for co-encapsulation of one cell and one particle in one droplet.

When the concentration of the samples in the dispersed phase fluid is low, the microfluidic device can be operated in the trapping mode to increase the concentration of the sample prior to encapsulation in droplets. This can be advantageous in achieving high encapsulation efficiency and through-put. For example, in some embodiments, to get high concentration of cells in the dispersed phase, the flow parameters (e.g., flow velocity and/or pressure of the continuous and the dispersed phases) can be adjusted using the fluid controller to generate vortices such that d_{gap} is less than the size (e.g., radius) of the cells so that all the particles or cells can be trapped in the vortices generated in the intersection region. The fluid controller can then be configured to release the trapped cells such that they are encapsulated in droplets within a short time interval (e.g., in less than 1 second).

In some embodiments, the flow in the microfluidic device may be pressure-driven. In one embodiment, the flow rates can be adjusted using a constant pressure source via high speed solenoid valves. The valves may be controlled by a custom-built lab view program. As used herein, the flow rate is equivalent to the fluid pressure or channel resistance. The flow rates may be adjusted to generate the vortices, or to modulate between trap and release modes by varying the ratio of ν_{d} to ν_{c} (ν_{d}/ν_{c}). In various embodiments, the flow rate of the continuous phase fluid streams can be about 2-10 times greater than the flow rate of the dispersed phase fluid stream. Alternatively, or in addition, the dispersed phase pressure to continuous phase pressure ratio (φ) may be modified to adjust between the various modes. The range of values for φ over which the device operates in the trapping mode and the range of values for φ over which the device operates in the release/encapsulation mode can vary depending on the size of the cells and particles used.

This application contemplates a passive, hydrodynamic technique that can trap cells and particles in independent vortices (e.g., two vortices) and then releasing the trapped cells and particles such that they are encapsulated in droplets. The size of the generated droplets can be adjusted to increase the probability that a single cell and single particle are encapsulated in a single droplet. As such, the droplet encapsulation efficiency can be increased to 30% or higher. Further details of hydrodynamic vortices can be found in co-owned U.S. patent application Ser. No. 16/005,533, the specifications of which are incorporated herein in their entirety by reference.

Acoustic Focusing

Referring to FIG. 3E, in an alternative embodiment, a vibrating air cavity (e.g. trapped air bubble in liquid) is positioned in the intersection region upstream from the orifice to disrupt particle-trapping vortices and facilitate cell/bead encapsulation. The vibrating air cavity can produce vortices that are in a direction opposite to the direction of the particle-trapping vortices produced as a result of an increase in rate of droplet generation and/or decrease in the size of the generated droplets.

As microfluidic droplet production rate is increased, and size is decreased, incoming cells and particles tend to become trapped in microvortices in the intersection region, instead of being encapsulated in the droplets. To disrupt the microvortices, an air cavity is integrated in the intersection region of a microfluidic device. The air cavity can be integrated upstream from the orifice of the droplet shearing region that generates the droplets. During droplet generation, the air trapped in the structure can be vibrated. The vibrations can produce vortices that flow counter to the microvortices, thereby reducing cell/bead trapping upstream from the orifice and improving encapsulation efficiency.

In some embodiments, the fluid controller can be configured to vibrate an air cavity placed upstream from the orifice. The air cavity can be vibrated to modulate the flow rate. The microfluidic device can further comprise a piezo-electric transducer configured to vibrate the air cavity. The air bubbles trapped in the sidewall lateral cavities can be excited using piezo-electric transducers. In such implementations, the vibrating air cavity/bubble can induce microstreaming of the fluid flowing through the microfluidic channel. Further details of acoustic focusing can be found in co-owned U.S. patent application Ser. No. 15/395,744, the specifications of which are incorporated herein in their entirety by reference.

Droplet Sorting

In further embodiments, the microfluidic device (100) may include a sorting module (170) operatively coupled to the output channel (150). The output channel (150) may be divided into a plurality of collection channels. The sorting module can direct droplets into one or more collection channels based on droplet content, sample type, or droplet size. The first collection channel may be further divided into a plurality of sub-collection channels. A second sorting module may be operatively coupled to the first collection channel, which directs the sample droplets into a specific sub-collection channel. In one example, the sample droplets may be sorted from empty droplets using the sorting module (170) operatively coupled to the output channel (150), which is bifurcated into a collection channel and a waste channel. For example, the sorting module (170) can direct the sample droplets (e.g. droplets encapsulating cells and particles) into a collection channel, and direct empty droplets into the waste channel.

In some embodiments, the sorting module may comprise one or more electrodes that sort the droplets by dielectrophoresis (DEP). In DEP, droplets are manipulated in non-uniform electric fields. The movement of droplets in DEP is based on the difference in polarizability between the droplets and the surrounding medium. The droplets carry electrical potential, and respond uniquely to the different frequencies. A non-uniform AC electrical field manipulates the motion of droplets by creating a polarizability gradient between the droplets and the suspending medium. When the droplets are exposed to this non-uniform electric field, two different forces occur between the droplets and surrounding medium leading to a resultant force that moves the droplet.

In other embodiments, the sorting module may comprise a lateral cavity acoustic transducer (LCAT) that sorts the droplets by LCAT sorting. Briefly, LCAT devices exploit the phenomenon of acoustic micro-streaming to manipulate fluid flow and suspended cells/particles within a microfluidic environment. Bubble-induced acoustic micro-streaming develops when bubbles trapped within a liquid phase oscillate when excited by a sound field. As described in US20140011291A1, the specification of which is incorporated herein in its entirety, a dead-end side channel or LCAT, which has a gas contained therein, is coupled to the outlet channel at the junction of the collection channels. A transducer is configured to apply an external source of acoustic energy. Actuation of the transducer effectuates symmetrical oscillation of a gas/liquid boundary at the junction. For instance, when the LCAT is in the OFF state, particles flow through to one collection. When the LCAT is in the ON state, the particles are deflected into another collection channel by the oscillation.

Film Technique

Coupled with detection methods, a controlled-release droplet platform could prove to be a powerful tool in life science, drug development, and any other field that requires efficient, cost-effective, precision experimentation on a population of cells exposed to a library of compounds. For example, fluorescent labeling and detection can allow real time quantification of molecular changes in the cell in response to the controlled modulation of the droplet's microenvironment. Furthermore, using fluorescent lifetime imaging microscopy (FLIM) also allows for non-invasive monitoring of the cell's metabolic changes in response to the controlled modulation of the droplet's microenvironment.

The present invention enables multiple types of high fidelity single cell studies (i.e. genotype, phenotype, and identification of other subcellular constituents). In some embodiments, monitoring of concentration inside droplets can be achieved using fluorescence (such as brightness increase over time as concentration in the droplet increases), enabling dynamic single cell studies as concentration changes. Alternatively, fluorescence lifetime imaging microscopy (FLIM) can be used to monitor cell metabolism changes as the concentration of the compound in the droplet increases over time. Combining this lab-on-a-chip microfluidic device with phasor FLIM enables noninvasive, label-free analysis of metabolism, secretion, and/or signaling at single cell resolution. In other embodiments, the phenotype of cells assessed by FLIM can be correlated with the genotype of cells by droplet sequencing (drop-seq) or droplet digital PCR (ddPCR).

In some embodiments, the present invention includes a FLIM microscopy system that can be used for FLIM analysis of droplets in a microfluidic chip. In one embodiment, the FLIM microscopy system may be made smaller and more portable. In another embodiment, the FLIM microscopy system is not integrated on the microfluidic chip. In a non-limiting example, downstream FLIM analysis may be used to characterize metabolic differences between proliferating and quiescent cells—a critical step towards label-free single cancer cell dormancy research. Media can be replaced with lysis buffer to lyse cells within droplets for gene sequencing applications.

In other embodiments, a droplet collection chamber can also be designed to facilitate other post-encapsulation functions (such as PCR). Downstream sorting may also be incorporated with FLIM. In one embodiment, an outlet of the droplet collection chamber may be fluidly coupled to a sorting module for sorting the droplets.

Droplet-based microfluidics enables more cost-effective multiplexed single cell analysis. Each droplet functions as a nano- to femtoliter microreactor. Previously, typical experiments of this nature required entire benchtops and/or robotic liquid handling systems. By implementing a droplet-based microfluidic device, the micro-environment inside the droplet can be changed due to controlled release particles already present in the droplet at the start or merging of droplets of different constituents at a droplet fusion junction downstream.

With the present invention, the co-encapsulation of a single cell and a controlled release particle in droplets not only allows the user to take full advantage of the versatility, efficiency, and economy of droplets as reaction vessels, but also allows users to study the temporal response of the cell to its changing microenvironment inside the droplet. The active, controlled temporal alteration of a droplet's microenvironment via a co-encapsulated release particle offers unprecedented precision, along with high throughput, not currently available. Downstream analysis and/or sorting may be incorporated after 1-1-1 encapsulation with the controlled release particle. Coupled with established detection methods, this controlled-release droplet platform could prove to be a powerful tool in life science, drug development, and any other field that requires efficient, cost-effective, precision experimentation on a population of cells exposed to a library of compounds. This capability would enable new data that can elucidate cell-level biological mechanisms and responses that can be studied, tuned, and utilized in the development of next generation medicines, foods, and other critical resources.

Applications

As such, the present invention may be used in diverse fields of research and analysis such as genomics, proteomics, emulsion microfluidics, pharmacology, agricultural biotechnology, etc. For example, the present invention may be used in high throughput screening of compounds, in conducting a consecutive series of chemical reactions within a droplet, in pharmacology for precise determination of toxicity levels of potential drug compounds for a variety of cell types, in agriculture biotechnology for precise compound delivery combined with single cell indexing/tracking ultimately for seed trait optimization, and in genomics, proteomics, single cell analysis for controlling the microenvironment in a droplet for high multiplexed, precision single cell studies, such as cell embryogenesis.

According to some embodiments, the present invention features a method of modifying an environment of a cell in a droplet (104). The method may comprise encapsulating the cell (102) and a controlled release particle (103) in the droplet (104), where the controlled release particle (103) comprises a cargo, and activating release of the cargo from the controlled release particle (103), which changes the environment in the droplet. Preferably, the droplet (104) remains intact during activation.

In other embodiments, the present invention features a drug screening method. The drug screening method may include the method of modifying an environment of a cell in a droplet (104) as previously described above. In this embodiment, the cargo is a drug compound to be analyzed for its effect on a cell.

According to some other embodiments, the present invention features a method of analyzing embryotic development. The method of analyzing embryotic development may comprise the method of modifying an environment of a cell in a droplet (104) as previously described above. In this embodiment, the cell is a plant or animal cell. The cargo may include nutrients or media for embryotic development and/or a compound that is being tested on its effects on embryotic development.

In some embodiments, the present invention features a method of analyzing an effect of a chemical or a biomolecule on a cell (102). The effect may be toxicity, binding, signaling, or cell development. The method may comprise encapsulating the cell (102) and a controlled release particle (103) in a droplet (104), where the controlled release particle (103) comprises the chemical or biomolecule, activating release of the chemical from the controlled release particle (103), and examining the effect of the chemical on the cell (102). Preferably, the droplet (104) remains intact during activation.

According to other embodiments, the present invention features a high-throughput screening method comprising preparing a droplet library comprising a plurality of droplets. The step of preparing the droplet library may comprise encapsulating in one droplet (104) a cell (102) and at least one controlled release particle (103) containing a chemical. The chemical of one controlled release particle (103) may be identical to or different from the chemical of another controlled release particle (103) that is encapsulated with a cell in another droplet. The screening method may further comprise activating release of the chemical from the controlled release particle (103) in the droplets (104), and examining the effect of the chemical on the cell (102). Preferably, the droplets (104) remain intact during activation. The method may be effective for screening a library of compounds.

In some embodiments, each droplet further comprises a second controlled release particle. The second controlled release particle can release its chemical at a different time from when the first controlled release particle releases its chemical to result in sequential reactions.

In conjunction with any of the methods described herein, the cell (102) may be an animal cell, plant cell, algae cell, bacterial cell, fungal cell, protoplast, pollen grain, microspore, or tetrad. Release of the cargo can directly affect the cell. For instance, release of the cargo may cause lysis of the cell or modulate cell development.

In some embodiments, the cargo may be released by a mechanism selected from ultrasound, magnetism, physical contact of the particle with the cell, light, pH, or temperature changes. In some embodiments, the cargo comprises one or more chemicals, biomolecules, or a combination thereof. The chemicals or biomolecules may have pharmacological activity, signaling activity, biological activity, pH activity, and/or biochemical activity. The chemicals or biomolecules may have a release profile indicative of concentration.

In some embodiments, the controlled release particle (103) has a solid core, a semi-solid core, a liquid core, or a gas core. In other embodiments, the controlled release particle (103) comprises multiple layers. Each layer can have a different chemical from that of the other layers. In some other embodiments, the controlled release particle (103) is bar-coded.

In further embodiments, the present invention features a method of analyzing nucleotides in an encapsulated droplet (104). The method may comprise encapsulating at least one cell (102) and at least one controlled release particle containing one or more reagents for sequencing (103) in the encapsulated droplet (104), activating release of the one or more reagents from the at least one controlled release particle, extracting nucleotides from the at least one cell, and sequencing the nucleotides of the at least one cell (102). In a preferred embodiment, the droplet (104) remains intact during activation.

In some embodiments, the cell (102) is a plant cell, an animal cell, or a microbial cell. In other embodiments, the nucleotides are RNA or DNA. In one embodiment, the method may further comprise encapsulating a lysis buffer in the encapsulated droplet (104). The lysis buffer may comprise a cell wall or cell membrane degrading reagent or enzyme. In one embodiment, the cell wall is a plant or microbial cell wall. In other embodiments, the reagents may comprise an RNA sequencing reagent, a neutralizing buffer for pH neutralization, and an RNA sequencing reagent inhibitor. In one embodiment, the RNA sequencing reagent may comprise reverse transcriptase. In some embodiments, the method may further comprise encapsulating sequencing barcode beads in the encapsulated droplet (104). In other embodiments, the at least one controlled release particle (103) is bar-coded.

In further embodiments, the present invention features methods for culturing plant cells in a droplet (104). The method may comprise encapsulating a plant cell and a controlled release particle (103) in a droplet (104), and activating release of the cargo from the controlled release particle (103). The droplet (104) may remain intact during activation and controlled release of the cargo changes a microenvironment of the droplet (104). Non-limiting examples of plant cells include microspores, protoplasts, walled cells, egg cells, or pollen. In further examples, the plant cells may have a modified genome. In other embodiments, the modified genome has been produced by genome editing facilitated by the release of the cargo. In some embodiments, the release of the cargo facilitates modifying a genome in the plant cell. Non-limiting examples of genome editing methods include CRISPR-CAS. In some embodiments, the cargo may be released by a mechanism selected from ultrasound, magnetism, physical contact of the particle with the cell, light, pH, or temperature changes.

In other embodiments, the present invention features a method for pollinating a plant using encapsulated pollen grains. In some embodiments, the method comprises encapsulating a pollen grain and a controlled release particle (103) in a droplet (104). The controlled release particle (103) may comprise a cargo. In further embodiments, the method comprises activating release of the cargo from the controlled release particle (103), and introducing the droplet (104) to a stigma of the plant to facilitate plant pollination. In preferred embodiments, the droplet (104) remains intact during activation and the release of the cargo changes an environment of the droplet (104). In some embodiments, the droplet is a hydrogel. In other embodiments, plant pollination is facilitated by activating controlled release of the cargo within the droplet by triggering the particle with light, ultrasound, magnets, changing the conductivity of the droplet, pH or temperature. In other embodiments, the release of the cargo facilitates pollen grain germination. As a non-limiting example, release of the cargo may chemically induce pollen tube formation.

In yet another embodiment, the present invention features a method for creating a double haploid cell using encapsulated microspores. In some embodiments, the method comprises encapsulating a haploid cell and a controlled release particle (103) in a droplet (104). The controlled release particle (103) may comprise a cargo. In some embodiments, the release of the cargo from the controlled release particle (103) is activated, facilitating chromosomal doubling. In preferred embodiments, the droplet (104) remains intact during activation. In some embodiments, the droplet (104) is a hydrogel. In other embodiments, the cargo is an embryogenesis enhancer or a doubling agent. Non-limiting examples of embryogenesis enhancers or doubling agents include colchicine, oryzalin, amiprophos-methyl (APM), pronamide, trifluralin, chlorpropham, or other mitotic inhibitors. In other embodiments, the cargo may be released by a mechanism selected from ultrasound, magnetism, physical contact of the particle with the cell, light, pH, or temperature changes.

In some embodiments, the present invention features a method for creating a microspore derived embryo using encapsulated microspores. The method may comprise encapsulating a haploid cell and a controlled release particle (103) in a droplet (104) and activating release of the cargo from the controlled release particle (103). The controlled release particle (103) may comprise a cargo, and the droplet (104) may remain intact during activation. In preferred embodiments, release of the cargo facilitates microspore embryogenesis development. In some embodiments, the droplet (104) is a hydrogel. In other embodiments, the cargo is an embryogenesis inducing factor. In some embodiments, the cargo may be released by a mechanism selected from ultrasound, magnetism, physical contact of the particle with the cell, light, pH, or temperature changes.

In other embodiments, the present invention features a method of delivering an agricultural chemical to a plant. The method may comprise encapsulating the agricultural chemical and a cargo in a droplet (104) and activating controlled release of the cargo from within the droplet (104). In preferred embodiments, the droplet (104) remains intact during activation, and the controlled release of the cargo changes a microenvironment of the droplet (104). Non-limiting examples of the agricultural chemical include a fertilizer, herbicide, insecticide, fungicide or biological. In some embodiments, the droplet (104) is a hydrogel. In further embodiments, activation of the cargo causes release of the agricultural chemical from the hydrogel. In some embodiments, the cargo may be released by a mechanism selected from ultrasound, magnetism, physical contact of the cargo with the cell, light, pH, changing a conductivity of the droplet (104), or temperature changes.

In any of the embodiments described herein, the droplet (104) may be a hydrogel. The hydrogel may be a natural hydrogel, a synthetic hydrogel, or a combination thereof. Examples of the hydrogels include, but are not limited to, agarose, alginates, methylcellulose, hyaluronan, or elastin-like peptides. The hydrogels may be, but are not limited to, pH-sensitive hydrogels, temperature sensitive hydrogels, electro-sensitive hydrogels and light responsive hydrogels.

### Example

The following are non-limiting examples of the present invention. It is to be understood that said examples are not intended to limit the present invention in any way. Equivalents or substitutes are within the scope of the present invention.

RNA Sequencing

The following is a non-limiting example of RNA sequencing in accordance with the present invention. A single-cell suspension was prepared from a source of interest (e.g. plant microspores), and bar-coded primers were prepared as bar-coded beads. The reaction buffer was used to form the droplet. The reaction buffer was beneficial for cell lysis and it protected RNA degradation (e.g., cell wall degrading enzymes, alkaline pH, and RNase inhibitors).

A first controlled release particle was prepared to contain the reaction buffer for neutralizing the lysis conditions and for achieving an optimal reaction condition for the reverse transcriptase reaction. A second controlled release particle was prepared to contain reverse transcriptase and reagents. This allowed for the synthesis of cDNA and addition of barcodes to the cDNA template. Each cell was individually co-encapsulated in a droplet with a bar-coded bead, lysis buffer, and the first and second controlled release particles. Following encapsulation, the cells in the droplets were lysed to release their mRNAs. Heating may be applied to assist cell lysis.

The first controlled release particle was activated by ultrasound to release the neutralizing and optimizing buffer for pH neutralization and for preventing enzymatic inhibitions for the next step of reverse transcription. The second controlled release particle was activated by light to release reverse transcriptase. The droplets were pooled for cDNA synthesis and for adding the barcodes by PCR amplification. A cDNA library that has a unique sequence identifier for each cell and each molecular by the barcodes was completed for sequencing.

Encapsulation of Plant Cells in Hydrogel Droplets for 3D Cell Culture

Without wishing to limit the invention to a particular theory or mechanism, compartmentalization of plant cells inside hydrogel microparticles may promote embryogenesis of microspores. During microspore preparation the viability drops significantly (roughly 1 out of every 1000 cells is viable for downstream experimentation). A main factor contributing to drop in viability is the adverse effect of unhealthy or dead cells on the growth and viability of the healthy cells. To significantly reduce and possibly eliminate this coupling effect, single microspores are encapsulated inside hydrogel “droplets,” i.e. 3D culture spheroids. Thus, microspores are isolated from each other in separate spheroids, while allowing on-going monitoring and fresh nutrient media exchange to keep them viable. Mostly, in plant tissue culture, the media composition (e.g. mineral elements, organic supplements, plant growth hormones, etc.) has a substantial impact on the response of tissue cultured plants. Furthermore, the developed platform allows for perfusion of encapsulated cells with different media compositions and thus can help better understand the effect of the media composition on the response of tissue cultured plants at single cell level.

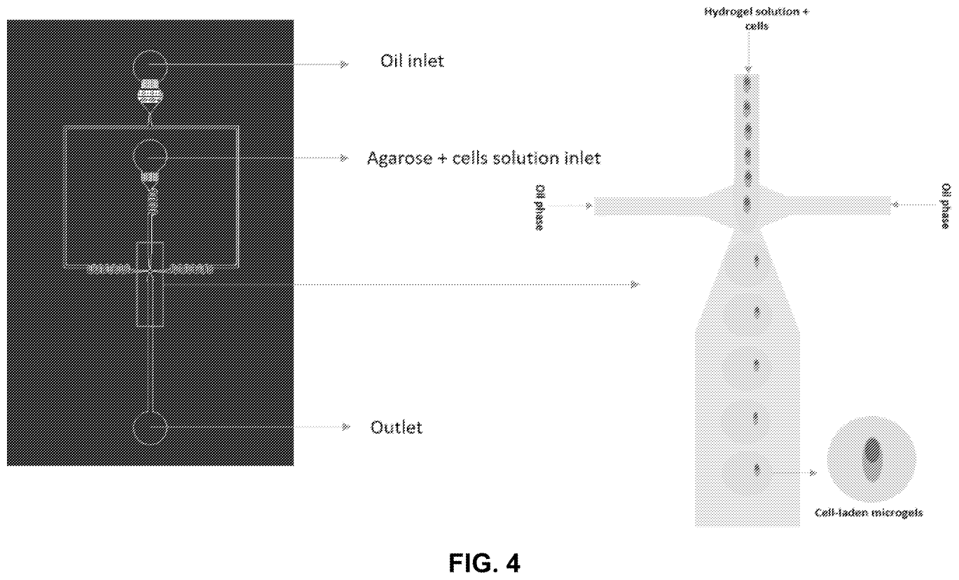

The present invention has the technique to generate hydrogel droplets using a focusing microfluidic device (FIG. 4). The hydrogel droplets can be generated at the rate of 2.5 Hz and they are uniform in size, with polydispersity index (average diameter/standard deviation) of less than 3%. Hydrogels diameter can range from 136-400 μm depending on the flow rates of the continuous and dispersed phase.

Agarose was chosen to develop single cell laden hydrogel droplets for plant tissue culture. Agarose is a natural polysaccharide derived from marine algae, which displays excellent biocompatibility, low toxicity and reversible polymerization. Moreover, agarose is crosslinked via thermally induced gelation which is advantageous since it does not have an adverse effect on the cell's viability. Other natural and synthetic hydrogels, or combinations thereof, may be used. These include, but are not limited to, alginates, methylcellulose, hyaluronan, and elastin-like peptides. The hydrogels may be, but are not limited to, pH-sensitive hydrogels, temperature sensitive hydrogels, electro-sensitive hydrogels and light responsive hydrogels.

Materials and Methods

Hydrogel Formation

The agarose solution was made with dissolving ultra low gelling temperature agarose (Sigma catalog number A5030) in the desired culture media to a final concentration of 1 wt % until completely melted. For the experiments involving cell encapsulation inside the microgels, microspore cells were resuspended inside the melted agarose. Light mineral oil was used as the continuous phase for the emulsification of the agarose solution. To stabilize the agarose droplets and prevent coalescence, 5% (v/v) of the surfactant Span80 was added to the mineral oil and 5% (v/v) of 10% pluronic F-68 solution to the agarose solution. The agarose solution and oil were each loaded in a syringe tipped with a 23 gauge needle (BD) to fit the tubing and is then delivered to the microfluidic device at a constant flow rate using a digitally controlled syringe pump (Pico Plus, Harvard Apparatus). The syringe containing the microbeads solution was placed vertically pointing downward at a level several tens of cm above the chip to help keep the beads flow inside the microfluidic chip. The agarose and oil flow rate ranged from 1-5 μL/min and 3-8 μL/min, respectively. The agarose droplets were collected into a vial placed on ice for the formation of agarose microgel.

Hydrogel Extraction

The agarose microgels were transferred to the 15 mL Falcon tubing and centrifuged at a 1000 rpm for 5 minutes, the supernatant oil phase was aspirated and replaced with the desired culture media, this process was repeated approximately 3 times until oil was completely removed.

Maize Microspore Culturing

Isolated maize microspores at a concentration of 10,000 to 60,000 cells/mL from a transgenic line with enhance embryogenesis capacity were encapsulated in a 1% agarose solution prepared in embryogenesis promoting media with 5% (v/v) of 10% pluronic F-68. The agarose and oil flow rates were 3 μL/min and 4 μL/min, respectively. Agarose microgels collected after 2 hours were considered one sample. After agarose encapsulation, the microspores were rinsed three times in embryogenesis promoting media to remove the surrounding mineral oil. After three rinses, the encapsulated microspores were resuspended in 60 mL of embryogenesis promoting media containing 0.1 ppm EMS for induction of embryogenesis. This material was then divided among 3 wells of a 12 well tissue culture plate (Falcon™ 353043). The microspores were then incubated in the dark at 28° C.

Canola Microspore Culturing

Isolated canola microspores from the Westar cultivar at a concentration of 20,000 cells/mL were encapsulated within 1% agarose prepared in embryogenesis promoting media with 5% (v/v) of 10% pluronic F-68. The flowrates were identical to the maize microspore conditions. Post encapsulation, the canola microspores collected after 1 hour of running the microfluidic device were rinsed three times in embryogenesis promoting media and resuspended in 10 mL of embryogenesis promoting media. This material was then divided among 6 wells of a 12 well tissue culture plate (Falcon™ 353043). The microspores were then incubated in the dark at 25° C.

Maize Pollen Encapsulation and Germination

Freshly collected maize pollen at a concentration of 3 mg/mL were encapsulated within 1% agarose prepared in pollen storage media with 5% (v/v) of 10% pluronic F-68. The agarose and oil flowrate ranged from 1-2 μL/min and 3-8 μL/min, respectively. Post encapsulation, the encapsulated pollen collected after 30 minutes of running the microfluidic device was rinsed three times in germination media and resuspended in 6 mL germination media. This material was then divided among 3 wells of a 12 well tissue culture plate (Falcon™ 353043). The material then incubated in the dark at room temperature for at least 12 hours before viewing.

Results and Discussion for Encapsulation of Microspores Inside the Hydrogels

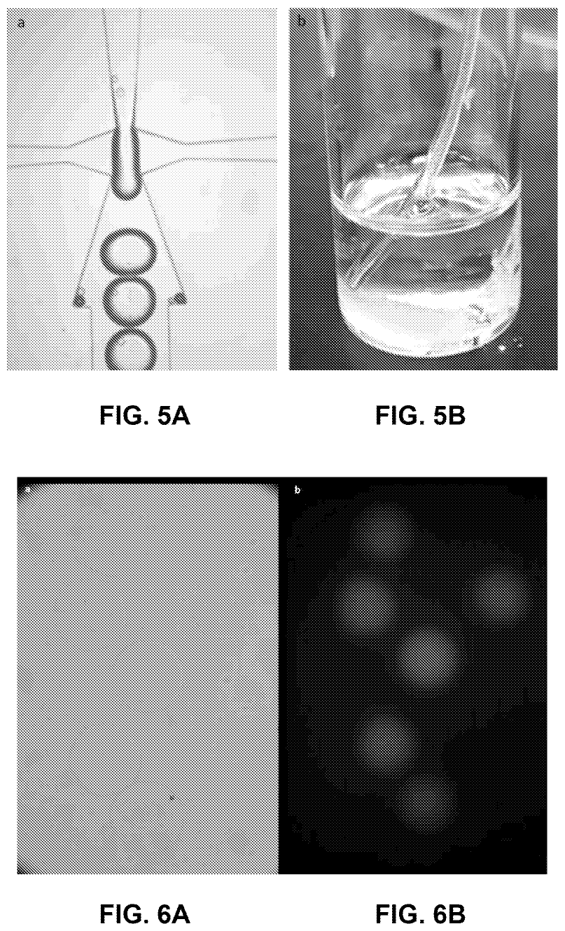

Agarose solution containing microspores was prepared as described in the methods section. FIGS. 5A-5B show snapshots from the microfluidic device during hydrogel generation, these images correspond to the case where the agarose solution and the oil phase have a flow rate of 2.5 μL/min and 4 μL/min, respectively. As it can be seen from these figures, highly monodisperse hydrogels (polydispersity index of <3%) with an average diameter of 357 μm, were generated with these flowrates. The number of hydrogels containing a single cell is dictated by Poisson's statistics. The amount of surfactant in continuous and dispersed phase were optimized to prevent any coalescence between the hydrogels. The hydrogels were collected inside a glass vial containing the same oil phase and were kept on ice to achieve agarose gelation. Moreover, clogging due to immature agarose gelation inside the microfluidic chip was not observed during the experiment. After the gelation the hydrogels were extracted from the oil phase and resuspended in culture media.

FIGS. 6A-6B show the bright field and fluorescent image of the hydrogels after resuspension in culture media. As can be seen from the figure, the monodispersity of the hydrogels was preserved, the hydrogels had an average diameter of 326 μm and a polydispersity index of only 3%. No damage was observed to the structure of microgels from the extraction process, only minor deformation in some of the microgels possibly due to the pipetting process. In fact, the preservation of the fluorescent probe inside the hydrogels confirms that the hydrogels were intact and did not suffer from structural damages.

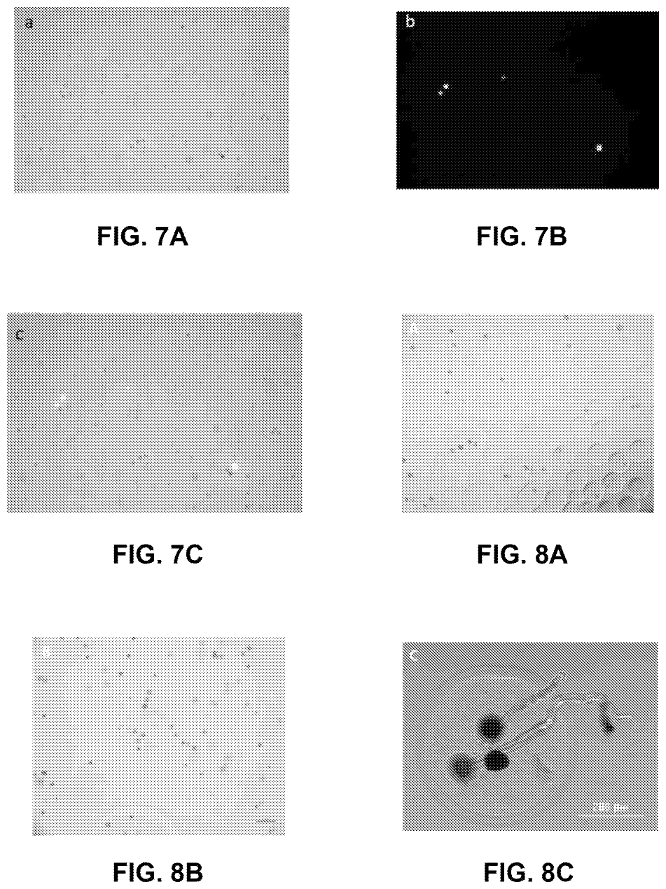

To confirm that the encapsulation and extraction processes do not have adverse effects on the microspores, viability FDA staining was performed after encapsulating microspores in hydrogels and resuspending them in embryogenesis promoting media. The hydrogels were incubated with FDA at concentration of 2 μl FDA/100 μl of solution. FIGS. 7A-7C shows gray scale and fluorescent image of the hydrogels after incubating with FDA and demonstrates successful FDA staining of the cells. The low viability of the microspores in this image is similar to the viability of the plant cells before encapsulation process, this confirms that the FDA was able to diffuse in to the extracted hydrogels which means that the developed protocol for extraction of the hydrogels from the oil and resuspension in culture media was successful and that the encapsulation and extraction processes did not have adverse effects on microspores viability.

Results and Discussion for Microspores Culture

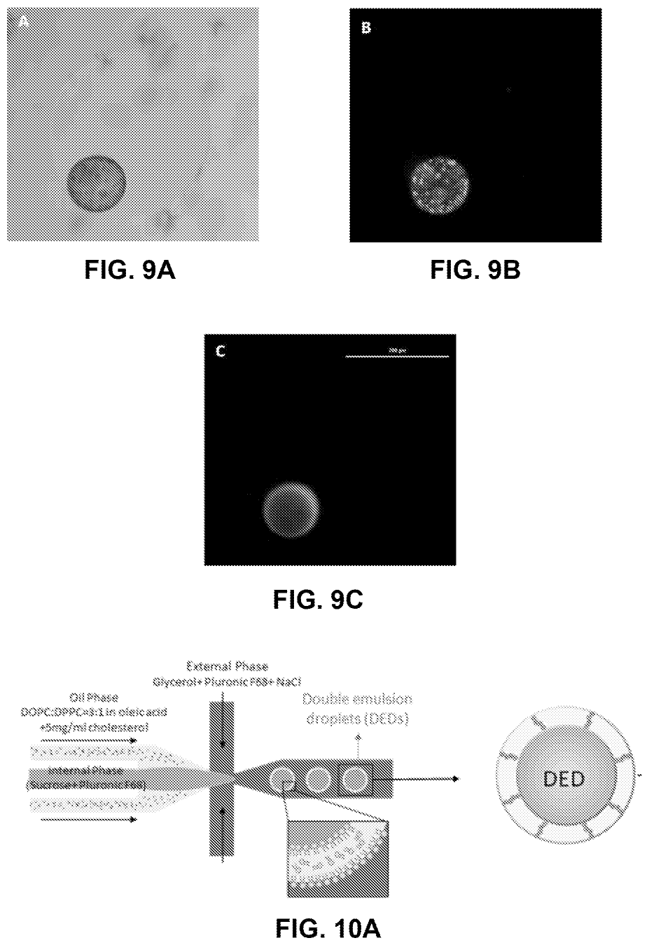

The microfluidic device has successfully encapsulated maize microspores, canola microspores, and maize pollen as illustrated in FIGS. 8A-8C. These encapsulated cells maintained viability post-encapsulation. When exposed to liquid pollen germination media, fresh maize pollen grains encapsulated in storage media germinated. This demonstrates the viability of the encapsulated pollen. Maize microspores encapsulated within a 1% agarose hydrogel remained viable for at least five days post-encapsulation as shown in FIGS. 9A-9C. In addition to remaining viable, a small portion of the encapsulated microspores enlarged. This was the first developmental change required before a microspore underwent embryogenesis.

Triggered Release of Reagents in Droplets with Encapsulated Plant Cells

The present invention has the technique to generate double emulsion droplets (DEDs) using a flow-focusing microfluidic device (FIG. 10A). The DEDs can be generated at the rate of 250 drops/s and they are uniform in size 25.13±0.77 μm (FIG. 10B).

Laser Induced Trigger Release

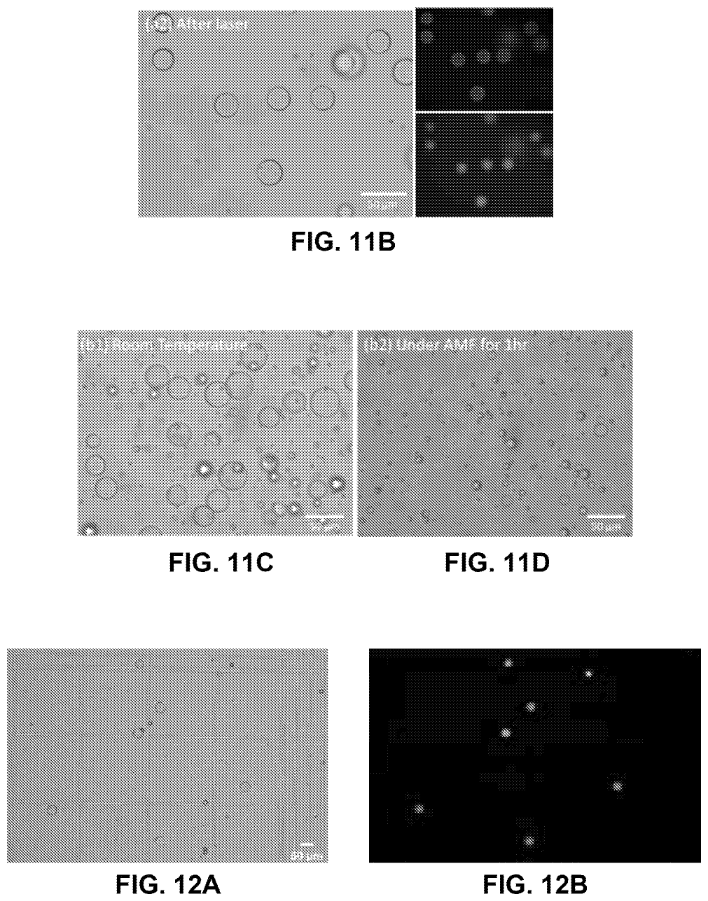

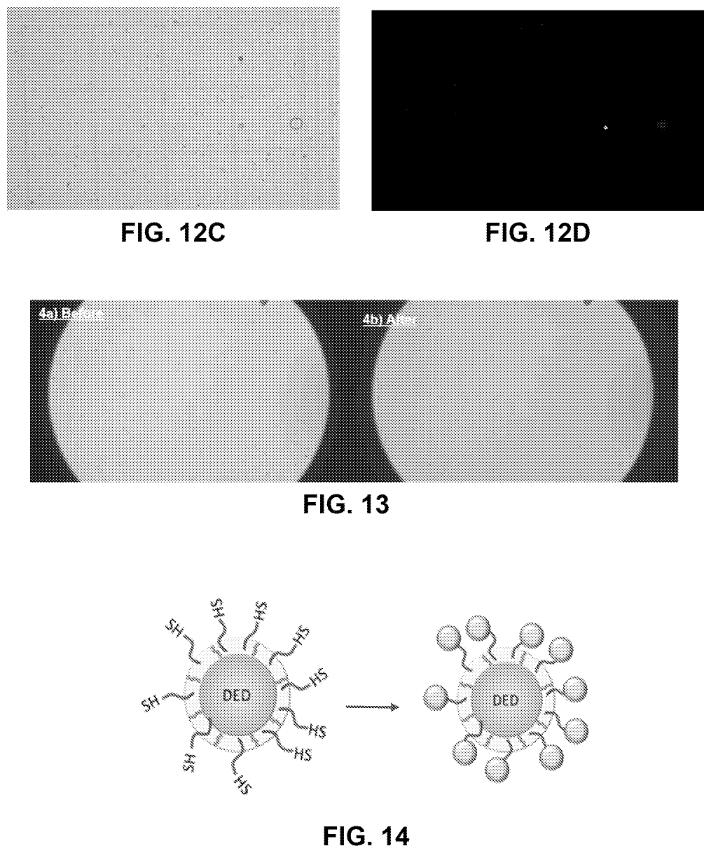

AuNPs develop surface plasmonic resonance (SPR) upon laser excitation, which heats up the droplet and causes it to break down. In preliminary experiments, the AuNPs were encapsulated either inside the inner core or on the oil shell of DEDs. After being exposed to laser for 50 minutes, the DEDs were still intact. Different laser wavelengths (405, 450 and 514 nm), particle sizes (5 nm and 30 nm), laser power (4.5 mW and 40 mW), and exposure time (15 min and 50 min) have been tested, but none of the conditions were able to break the droplets (FIG. 11A). In this regard, the aim was to tether AuNPs on the surface of DEDs so the plasmonic or magnetic energy can reach the particles more easily.

AuNPs were tethered on the surface of droplets using thiol-gold reaction conjugation. After 10 minutes of laser, the number of AuNPs-modified DEDs reduced to 27% of that at time 0. It was inferred that the AuNPs experienced plasmonic resonance upon the laser exposure, which induced the local heat on the surface, thus breaking the droplet. Without laser exposure, the AuNPs-DEDs did not have significant decrease in number, with 86% remaining after 10 minutes. To confirm whether the laser itself heats up the DEDs without the use of AuNPs, laser was applied to the plain DEDs for the same amount of time. The result showed that almost all (97%) DEDs remained, proving that the droplet breakage was mainly caused by the plasmonic resonance of AuNPs.

TABLE 1
The number of DEDs before and after laser exposure.

| | | | |
|---|---|---|---|
| | Control group | Exposure group | Exposure group |
| Time | DED + AuNPs | DED + AuNPs | DED |

| | | | | |
|---|---|---|---|---|
| 0 | min | 100% | — | 100% |
| 10 | min | 86% | 27% | 97% |

Magnetic Field Induced Trigger Release

In the presence of an alternating magnetic field (AFM), super magnetic nanoparticles dissipated heat through hysteresis loss which is presented in terms of the Néelian and Brownian relaxation. In Néelian relaxation the heating occurs due to the energy loss because of the rotation of individual magnetic moments within the particles, while the Brownian relaxation refers to the heat generated via the physical rotation of particles due to the alignment process of magnetic moments with the external applied magnetic field. Several parameters affected the amount of heat loss through this process. The type and size and concentration of the MNPs affected the generation and distribution of heat. Dynamic viscosity of the medium influenced the amount of heat generated by Brownian motion. Moreover, the parameters of applied magnetic field such as strength and frequency affected the magnitude of heat generated by MNPs.

Initially, the Iron Oxide MNPs (50-100 nm in diameter) were encapsulated on the oil shell of DEDs and the DEDs were exposed to AFM for 1 hour. As a result, a low percentage of DEDs broke down while many of DEDs remained intact. (FIG. 11B) While these results showed some promise of triggered release, the response time needed to be reduced. The high response time may be due to the following reasons: 1) The nanoparticles that were used are big in size and polydisperse; 2) The viscosity of the oil phase was high, which reduces the amount of heat generated by Brownian motion; 3) The droplets dewet which decreases the droplet stability and causes the magnetic nanoparticles to be unevenly distributed; 4) The magnetic nanoparticles were not functionalized and thus had a high tendency to aggregate together; 5) The strength and frequency of the induction heating used was not customizable

To address these issues, monodisperse (5 nm) neutravidin-Iron MNPs were conjugated on the surface of the biotinylated DEDs. This was advantageous since the MNPs were now located on the surface of the DEDs, where the solution was less viscous compared to the oil phase and their distribution was more even among all the particles. In addition, dewetting of the droplets was no longer a problem. In fact, by selectively dewetting the droplets before AFM exposure, the MNPs were adjacent to the lipid bilayer and it resulted in more efficient breakage and release of the cargo. FIG. 13 shows the image of the DEDs before and after AFM exposure. The results show that after ˜1 hour AFM exposure at a frequency of 46.43 KHz and current of 26.4 amps, 62% of the DEDs were broken (see Table 2). Although the percentage of DEDs that were broken over the 1 hour AFM exposure period were improved, the response time was still high, which can be optimized by modifying the frequency and strength of the magnetic field. In some embodiments, the AFM unit is primary and the frequency and strength of the magnetic field are preset. In other embodiments, the AFM unit may be customized.

TABLE 2
The number of DEDs before and after AFM exposure.

| | | |
|---|---|---|
| Time | Control group | Exposure group |

| 0 | | 1.3 × 10⁵ | — |
|---|---|---|---|
| 30 | min | 9.5 × 10⁴ | 9.7 × 10⁴ |
| 1 | hour | 1.2 × 10⁵ | 4.5 × 10⁴ |

Materials and Methods

Droplet Generation

The DEDs were generated using a microfluidic flow-focusing channel, with an orifice size of 15 μm. The inner aqueous phase was composed of 250 mM sucrose with 1% Pluronic F68% surfactant. The middle oil phase comprised 7.5 mg/mL DOPC, 2.5 mg/mL DPPC and 5 mg/mL cholesterol dissolved in oleic acid. For droplet functionalization, 10 mol % of thiolated lipids (16:0 Ptd Thioethanol) was added to the lipid mixture for droplet generation. The continuous phase contained 15% glycerol, 6% Pluronic F68 and 125 mM NaCl for osmolarity balance. DEDs were preserved in the continuous phase.

Droplet Functionalization

5 nm AuNPs were incubated with thiolated-DEDs at the ratio of NPs(mg):Thiol group (mmol)=135:1. After 24 hours of incubation at room temperature, DEDs were washed by centrifugation, aspirating out the supernatant, and filling fresh continuous phase solution, to remove excessive or loosely bonded AuNPs. The MNPs-DEDs were fabricated by conjugating neutravidin-MNPs (5 nm) to the biotinylated DEDs. Biotinylated DEDs were generated by adding 10 mol % biotinylated lipid (DSPE-PEG(2000)-Biotin) to the lipid mixture. Neutravidin-MPs were incubated with the DEDs room temperature for an hour, and DEDs were washed as described above. The molar ratio of neutravidin to biotin (on the DEDs) was 5:1.

Droplet Breakdown Upon Trigger

For laser-induced trigger, 100 μL AuNPs-DEDs (105 drops/mL) were transferred to one well in a 96-well plate. Laser light (450 nm, 4 mW) was fixed on top of the well and the DEDs were exposed to laser for 10 min. The concentration of DEDs was recorded before and after exposure.

For alternating magnetic field trigger, 100 μL MNPs-DEDs (105 drops/mL) were transferred to PCR reaction tubes and placed inside the coil of an induction heating unit. The DEDs were exposed to AFM for 1 hour. The concentration of the DEDs was calculated before and after every 30 minutes exposure. Control groups were included to make sure that droplet breakage was due to the AFM exposure rather than inherent instability of the droplets.

As used herein, the term “about” refers to plus or minus 10% of the referenced number. Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the below claims are solely for ease of examination of this patent application, and are exemplary, and are not intended in any way to limit the scope of the claims to the particular features having the corresponding reference numbers in the drawings. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of modifying an environment of a cell in a droplet (104), the method comprising:
a. encapsulating the cell (102) and a controlled release particle (103) in the droplet (104), wherein the controlled release particle (103) comprises a cargo; and
b. activating release of the cargo from the controlled release particle (103), wherein the droplet (104) remains intact during activation, wherein release of the cargo changes the environment in the droplet.

2. The method of claim 1, wherein the cell (102) is an animal cell, plant cell, algae cell, bacterial cell, fungal cell, protoplast, pollen grain, microspore, or tetrad.

3. The method of claim 1, wherein the cargo comprises one or more chemicals or biomolecules.

4. The method of claim 3, wherein the one or more chemicals or biomolecules have pharmacological activity, signaling activity, biological activity, pH activity, or biochemical activity.

5. (canceled)

6. The method of claim 1, wherein the cargo is released by a mechanism selected from ultrasound, magnetism, physical contact of the particle with the cell, light, pH, or temperature changes.

7. The method of claim 1, wherein the controlled release particle (103) has a solid core, a semi-solid core, a liquid core, or a gas core.

8. The method of claim 1, wherein the controlled release particle (103) comprises multiple layers, each laver having a same or different chemical from that of the other layers.

9. The method of claim 1, wherein release of the cargo affects the cell.

10. The method of claim 9, wherein release of the cargo causes lysis of the cell.

11. The method of claim 9, wherein release of the cargo modulates cell development.

12. (canceled)

13. A drug screening method comprising the method of claim 1, wherein the cargo is a drug compound to be analyzed for its effect on a cell.

14-40. (canceled)

41. A method of culturing plant cells, the method comprising:
a. encapsulating a plant cell and a controlled release particle (103) in a droplet (104), wherein the controlled release particle (103) comprises a cargo; and
b. activating release of the cargo from the controlled release particle (103);
wherein the droplet (104) remains intact during activation, and wherein the release of the cargo changes a microenvironment of the droplet (104).

42. The method of claim 41, wherein the plant cells are microspores, protoplasts, walled cells, egg cells, or pollen.

43. The method of claim 41, wherein the plant cell has a modified genome.

44. The method of claim 43, wherein the modified genome has been produced by genome editing facilitated by cargo release.

45-51. (canceled)

52. A method for creating a microspore derived embryo using encapsulated microspores, the method comprising:
a. encapsulating a haploid cell and a controlled release particle (103) in a droplet (104), wherein the controlled release particle (103) comprises a cargo; and
b. activating release of the cargo from the controlled release particle (103) wherein the droplet (104) remains intact during activation, wherein release of the cargo facilitates microspore embryogenesis development.

53. The method of claim 52, wherein the droplet (104) is a hydrogel.

54. The method of claim 52, wherein the cargo is an embryogenesis inducing factor.

55-58. (canceled)

59. The method of claim 44, wherein the genome editing comprises a CRISPR-CAS method.

60. The method of claim 59, wherein the plant cells are pollen.
